(19) United States
(12) Patent Application Publication
(10) Pub. No.: US 2026/0109672 A1
(43) Pub. Date: Apr. 23, 2026

(54) SARS-COV-2 MPRO INHIBITORS AND USES THEREOF

(71) Applicant: NXERA PHARMA UK LIMITED, Cambridge, Cambridgeshire (GB)

(72) Inventors: Miles Stuart CONGREVE, Cambridge (GB); John Andrew CHRISTOPHER, Cambridge (GB); Mark PICKWORTH, Cambridge (GB); Chris DE GRAAF, Cambridge (GB); Alicia Perez HIGUERUELO, Cambridge (GB); Jonathan Stephen MASON, Cambridge (GB)

(73) Assignee: NXERA PHARMA UK LIMITED, Cambridge (GB)

(21) Appl. No.: 19/117,628

(22) PCT Filed: Oct. 5, 2023
(86) PCT No.: PCT/EP2023/077644

§ 371 Date: Apr. 1, 2025

(30) Foreign Application Priority Data
Oct. 5, 2022 (GB) 2214644.3

(51) Int. Cl. C07D 209/52; A61K 31/403; A61K 31/427; A61K 31/454; A61K 31/513; A61P 31/14; C07D 401/12; C07D 403/12

(52) U.S. Cl. CPC C07D 209/52; A61K 31/403; A61K 31/427; A61K 31/454; A61K 31/513; A61P 31/14; C07D 401/12; C07D 403/12

(57) ABSTRACT

This application relates to novel compounds and their use as SARS-CoV-2 Main Protease (Mpro) inhibitors. Compounds described herein may be useful in the treatment of SARS-CoV-2 and related viruses and disorders associated with SARS-CoV-2: Mpro. The application is also directed to pharmaceutical compositions comprising these compounds and the manufacture and use of these compounds and compositions in the treatment of SARS-CoV-2 and related viruses and disorders associated with SARS-CoV-2: Mpro. The compounds and compositions may be useful in preventing death or complications arising due to chronic underlying conditions or comorbidities in patients infected with SARS-CoV-2 and related viruses.

### FIELD OF THE INVENTION

This application relates to novel compounds and their use as SARS-CoV-2 Main Protease (Mpro) inhibitors. Compounds described herein may be useful in the treatment of SARS-CoV-2 and related viruses and disorders associated with SARS-CoV-2: Mpro. The application is also directed to pharmaceutical compositions comprising these compounds and the manufacture and use of these compounds and compositions in the treatment of SARS-CoV-2 and related viruses and disorders associated with SARS-CoV-2: Mpro. The compounds and compositions may be useful in preventing death or complications arising due to chronic underlying conditions or comorbidities in patients infected with SARS-CoV-2 and related viruses.

### BACKGROUND

Coronaviruses have long existed in nature and have made zoonotic transmission to humans, generally causing mild respiratory illnesses such as the common cold upon infection. However, in the last two decades outbreaks of novel human coronavirus infections that cause severe respiratory illness have presented a major global health concern. This includes the severe acute respiratory syndrome coronavirus (SARS-CoV) outbreak in 2002-2004, the Middle East respiratory syndrome coronavirus (MERS-CoV) outbreak in 2012-2015 and severe acute respiratory syndrome coronavirus 2 (SARS-CoV-2), the most recently emerged strain of coronavirus, that was identified in Wuhan, China, in 2019 and is the aetiological agent responsible for the 2019-2020 viral pneumonia outbreak of coronavirus disease 2019 (COVID-19). Despite the tragic and widespread effects of these sudden occurrences and the periodic emergence of novel human coronaviruses increasing the potential for future outbreaks, we do not yet have validated antiviral treatments targeting coronavirus infections.

SARS-CoV-2 packages a large RNA genome of ˜30 kb, two-thirds of which encodes for the two polyproteins pp1a and pp1b (Hegyi et al. Journal of General Virology 83 (3): 595-99). These polyproteins are processed into 16 non-structural proteins (nsps) that are liberated from the long polypeptide chains by two viral cysteine proteases, the papain-like protease (nsp3) and the 3C-like protease (nsp5). The latter species, also referred to as the main protease (Mpro), cleaves the viral polyproteins at eleven sites to generate twelve non-structural proteins (nsp5-16). Included in these nsps are those involved in the replication and transcription machinery such as the RNA-dependent RNA polymerase (nsp12) and helicase (nsp13). The essential role Mpro plays in viral replication has been demonstrated in mutagenesis experiments (Kim et al. Virology 208 (1): 1-8; Stobart et al. Journal of Virology 86 (9): 4801-10), which makes it an attractive target for the design of inhibitors to treat coronavirus infection. Furthermore, there are no human proteases with similar cleavage specificity and therefore selective inhibitors of Mpro are highly likely to be non-toxic (Anand et al. 2003. Science 300 (5626): 1763-67).

The use of protease inhibitors for the treatment of viral diseases is well precedented (Bacon et al. The New England Journal of Medicine 364 (13): 1207-17) and the similarity of the SARS-CoV-2 Mpro active site to other viral proteases has driven efforts to identify clinically approved drugs that could be repurposed for the treatment of COVID-19 (Riva et al. Nature, 586: 113-119). Screening of a selection of 18 viral protease inhibitors designed for the treatment of human immunodeficiency virus (HIV) and Hepatitis C virus (HCV) identified the anti-HCV drug boceprevir and the pre-clinical inhibitor against feline infectious peritonitis virus (FIPV) GC376 as inhibitors of SARS-CoV-2 Mpro (Fu et al. Nature Communications 11 (1): 4417). While GC376 showed a more potent inhibition efficacy of recombinant protease activity (IC₅₀=0.15 μM) than boceprevir (IC₅₀=8 μM), GC376 has shown side effects in trials performed in cats raising potential safety concerns (Pedersen et al. Journal of Feline Medicine and Surgery 20 (4): 378-92). Boceprevir was also identified as an inhibitor of SARS-CoV-2 Mpro alongside telaprevir in a different study, albeit both drugs inhibited SARS-CoV-2 Mpro with IC₅₀ values of >1 μM (Anson et al. 2020. doi:10.21203/rs.3.rs-26344/v1). In addition to SARS-CoV-2 Mpro, the inhibitory efficacy of boceprevir and telaprevir was also assessed at Mpro proteases from eight other coronaviruses including SARS, MERS, HKU1, HKU4, HKU5, NL63, FIPV and IBV. Within this selection boceprevir was able to inhibit all coronavirus proteases tested except NL63 and a similarly broad spectrum of activity was shown for telaprevir with inhibitory activity shown at SARS, HKU4, HKU5, NL63 and IBV. While the antiviral activity of these drugs at SARS-CoV-2 Mpro is not sufficient for clinical development, their ability to inhibit a broad range of proteases highlights the potential for the design of broad-spectrum antiviral drugs able to treat not only SARS-CoV-2 infection but also other human coronaviruses and potentially novel coronaviruses that could emerge in the future.

The sequence similarly between SARS-CoV and SARS-CoV-2 Mpro active sites was also exploited in the identification of the SARS-CoV-2 Mpro inhibitor PF-07304814, a phosphate prodrug of PF-00835231 which was originally designed for the treatment of SARS-CoV (Boras et al. BioRxiv, 2020.09.12.293498). PF-00835231 inhibited SARS-CoV-2 Mpro with a Kᵢ of 0.27 nM and displayed broad inhibitory activity against ten further coronavirus strains with Kᵢ values of 0.03-4 nM. This translated into ˜1 μM activity in cell-based live virus assays. The activity of PF-00835231 in combination with remdesivir, a nucleoside RNA-dependent RNA polymerase inhibitor, was also evaluated as antiviral agents that target different aspects of the viral replication process can yield synergistic effects in combination. Indeed, PF-00835231 and remdesivir displayed either synergistic or additive effects in a cell-based antiviral assay, which suggests that the combination of Mpro inhibitors with antivirals with other modes of actions could show clinical benefit.

In 2020 the crystal structure of SARS-CoV-2 Mpro in complex with N3 (a Michael acceptor inhibitor) was published (Jin et al. Nature 582 (7811): 289-93), thereby enabling virtual screening and structure-based drug design (SBDD) for inhibitors of SARS-CoV-2 Mpro. Such SBDD efforts included the design of peptidomimetic α-ketoamides as broad-spectrum inhibitors of coronaviruses and enteroviruses with the two most promising inhibitors showing 0.71-12.27 μM IC₅₀ values in recombinant inhibition assays for proteases from enteroviruses EV-A71 and CVB3 as well as coronaviruses SARS-CoV and NL63 (Zhang et al. 2020. Journal of Medicinal Chemistry 63 (9): 4562-4578). The activity observed in the recombinant protease assays broadly matched antiviral activity in cell-based live virus assays with IC₅₀ values within 10-fold in both systems, suggesting that good activity in the protease inhibition assay is a good indicator of antiviral activity.

Currently, there are no targeted therapeutic agents for the treatment of COVID-19, and effective treatment options remain very limited. Despite much ongoing research activity and numerous clinical trials in progress, only remdesivir and favipiravir have been approved in selected countries for limited use to treat SARS-CoV-2 infection but show only modest effects (Zhou et al. ACS Pharmacology & Translational Science 3 (5): 813-834). There exists a need for targeted therapeutic agents for the treatment of SARS-CoV-2 infection and for the reasons outlined above SARS-CoV-2 Mpro represents an attractive drug target for SARS-CoV-2. The compounds disclosed herein are shown to be inhibitors of SARS-CoV-2 Mpro and therefore represent potential candidates for the treatment of coronavirus infection and associated disorders including but not limited to COVID-19.

### SUMMARY OF INVENTION

The present invention provides compounds having activity as SARS-CoV-2: Mpro inhibitors. Therefore, in a first aspect of the invention, the compounds are of Formula (I′)

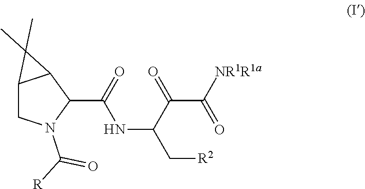

or a salt, solvate, hydrate, N-oxide or prodrug thereof, wherein:
   R¹ and R^{1a} are independently H; a C₁₋₆ saturated hydrocarbon group optionally containing a cycloalkyl group and optionally substituted with 1 to 6 halo; or a benzyl group optionally substituted with 1 to 6 halo; or R¹ and R^{1a} are linked, together with the nitrogen to which they are attached, to form a 3-6 membered saturated ring optionally containing an additional heteroatom and optionally substituted with 1 to 6 halo;
   R² is C₃₋₆ cycloalkyl which is optionally substituted with one or more oxo, hydroxy and halo groups;
   R represents

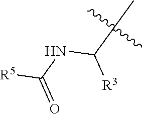

R³ is selected from
(i) a saturated group containing 1 to 6 carbon atoms and optionally containing a cycloalkyl group; or
(ii) a saturated ring containing an oxygen or nitrogen heteroatom, said saturated group or ring being optionally substituted with one or more substituents chosen from fluorine, hydroxy or methoxy; or
R³ is —CH₂aryl; —CH(CH₃)aryl; or —C(CH₃)₂aryl; and
R⁵ is a C₁₋₈ hydrocarbon group, optionally containing one or more rings or a double bond and which is optionally substituted with one or more groups selected from halo; cyano; hydroxy; methoxy; amino; and a cycloalkyl, heterocycloalkyl, phenyl and heteroaryl; or
R represents -L-A
   wherein L is —CR⁷═CR⁸—, —CHR⁹—CHR¹⁰— or —O—CHR¹¹—;
   R⁷ to R¹¹ are independently H, —(CH₂)ₘCO₂R¹² or C₁₋₃ alkyl optionally substituted with 1 to 6 fluorine atoms; or
   R⁹ and R¹⁰ are joined to form a cyclopropyl;
   A is phenyl or heteroaryl, optionally substituted with one or more groups selected from halo, —CN, —CO₂R¹³, —OR¹³, —SO₂R¹³, —SONHR¹³, —OSO₂R¹³, —PO(R¹³)₂, —SF₅, C₁₋₃ alkyl and C₁₋₃ haloalkyl;
   R¹² and R¹³ are independently selected from H, C₁₋₃ alkyl, and C₁₋₃ haloalkyl; and
   m is independently 0 to 3.

It is preferable that when R² is a C₃₋₅ saturated hydrocarbon group containing a cycloalkyl group which is unsubstituted or substituted with one or more substituents chosen from fluorine or hydroxyl, then
R represents

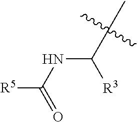

in which R⁵ is methyl, optionally substituted with one or more substituents chosen from halo; cyano; hydroxy; methoxy; amino; with R³ as defined herein; or
R represents -L-A as defined herein.

It is also preferrable that said compounds of the invention are not
(1R,2S,5S)—N-(4-Amino-1-cyclopropyl-3,4-dioxobutan-2-yl)-3-((S)-3,3-dimethyl-2-(2,2,2-trifluoroacetamido)butanoyl)-6,6-dimethyl-3-azabicyclo[3.1.0]hexane-2-carboxamide or a salt thereof; or
(1R,2S,5S)—N-(4-(Azetidin-1-yl)-1-cyclopropyl-3,4-dioxobutan-2-yl)-3-((S)-3,3-dimethyl-2-(2,2,2-trifluoroacetamido)butanoyl)-6,6-dimethyl-3-azabicyclo[3.1.0]hexane-2-carboxamide or a salt thereof.

In a second aspect of the invention, there is provided a pharmaceutical composition comprising a compound of the invention, including a salt, solvate, hydrate, N-oxide or prodrug thereof, and a pharmaceutically acceptable excipient.

In a third aspect of the invention, there is provided a compound of the invention, or a pharmaceutical composition comprising said compound, for use in the treatment of SARS-CoV-2 or in the treatment of disorders associated with SARS-CoV-2.

In a fourth aspect of the invention, there is provided a use of a compound of the invention in the manufacture of a medicament for the treatment of SARS-CoV-2 or a disorder associated with SARS-CoV-2.

In a fifth aspect of the invention, there is provided a method of treating a disease or disorder susceptible to SARS-CoV-2 Mpro inhibition in a subject, said method comprising administering a pharmaceutically effective amount of a compound of the invention, or salt, solvate, hydrate, N-oxide or prodrug thereof. A feature of this aspect is that the disease or disorder is SARS-CoV-2 or a disorder associated with SARS-CoV-2.

### DETAILED DESCRIPTION

The invention relates to the use of compounds as inhibitors of SARS-CoV-2: Mpro. The invention further relates to the use of novel compounds in the manufacture of medicaments for use as SARS-CoV-2: Mpro inhibitors. The invention further relates to compounds, compositions and medicaments that may be useful in the treatment of SARS-CoV-2 and related viruses or conditions or symptoms related thereto.

The compounds are of Formula (I′) as defined herein. Also described herein are compounds of Formula (I)

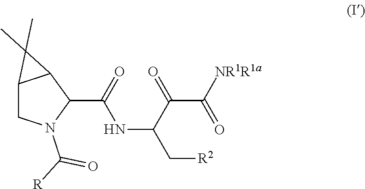

or a salt, solvate, hydrate, N-oxide or prodrug thereof, wherein:
   R¹ and R^{1a} are independently H; a C₁₋₆ saturated hydrocarbon group optionally containing a cycloalkyl group and optionally substituted with 1 to 6 halo; or a benzyl group optionally substituted with 1 to 6 halo; or R¹ and R^{1a} are linked, together with the nitrogen to which they are attached, to form a 3 to 6 membered saturated ring optionally containing an additional heteroatom and optionally substituted with 1 to 6 halo;
   R² is selected from a C₃₋₆ cycloalkyl, C₁₋₄ alkyl and saturated 3- to 6-membered heterocyclic ring (preferably the saturated 3- to 6-membered heterocyclic ring does not contain an oxygen atom in the ring), each of which are optionally substituted with one or more oxo, hydroxy and halo groups; or R² is —(CH₂)ₚCONHR⁶, or R² is —(CH₂)ₚCO₂R⁶;
   R⁶ is selected from H, C₁₋₃ alkyl, and C₁₋₃ haloalkyl;
   R represents

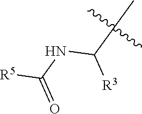

R³ is selected from
(i) a saturated group containing 1 to 6 carbon atoms and optionally containing a cycloalkyl group; or
(ii) a saturated ring containing an oxygen or nitrogen heteroatom, said saturated group or ring being optionally substituted with one or more substituents chosen from fluorine, hydroxy or methoxy; or
R³ is —CH₂aryl; —CH(CH₃)aryl; or —C(CH₃)₂aryl; and
R⁵ is a C₁₋₈ hydrocarbon group, optionally containing one or more rings or a double bond and which is optionally substituted with one or more groups selected from halo; cyano; hydroxy; methoxy; amino; and a cycloalkyl, heterocycloalkyl, aryl and heteroaryl; or
R represents -L-A
   wherein L is —CR⁷═CR⁸—, —CHR⁹—CHR¹⁰— or —O—CHR¹¹—;
   R⁷ to R¹¹ are independently H, —(CH₂)ₘCO₂R¹² or C₁₋₃ alkyl optionally substituted with 1 to 6 fluorine atoms; or
   R⁹ and R¹⁰ are joined to form a cyclopropyl;
   A is a phenyl or heteroaryl, optionally substituted with one or more groups selected from halo, —CN, —CO₂R¹³, —OR¹³, —SO₂R¹³, —SONHR¹³, —OSO₂R¹³, —PO(R¹³)₂, —SF₅, C₁₋₃ alkyl and C₁₋₃ haloalkyl;
   R¹² and R¹³ are independently selected from H, C₁₋₃ alkyl, and C₁₋₃ haloalkyl; and
   m is independently 0 to 3;
and wherein when R² is a C₃₋₅ saturated hydrocarbon group containing a cycloalkyl group which is unsubstituted or substituted with one or more substituents chosen from fluorine or hydroxyl, then
   R represents

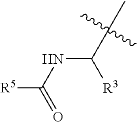

in which R⁵ is methyl, optionally substituted with one or more substituents chosen from halo; cyano; hydroxy; methoxy; amino; with R³ as defined herein; or
R represents -L-A as defined herein; and
wherein the compound of Formula (I) is not
(1R,2S,5S)—N-(4-Amino-1-cyclopropyl-3,4-dioxobutan-2-yl)-3-((S)-3,3-dimethyl-2-(2,2,2-trifluoroacetamido)butanoyl)-6,6-dimethyl-3-azabicyclo[3.1.0]hexane-2-carboxamide or a salt thereof; or
(1R,2S,5S)—N-(4-(Azetidin-1-yl)-1-cyclopropyl-3,4-dioxobutan-2-yl)-3-((S)-3,3-dimethyl-2-(2,2,2-trifluoroacetamido)butanoyl)-6,6-dimethyl-3-azabicyclo[3.1.0]hexane-2-carboxamide or a salt thereof.

The compounds of formula (I′) or (I) may optionally be compounds according to formula (Ii) below:

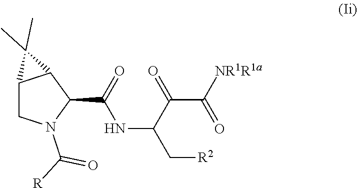

wherein the stereochemistry is as shown above, and the substituents are as defined elsewhere herein.

The term “C₁₋₈ hydrocarbon group” is a group that consists of 1, 2, 3, 4, 5, 6, 7, or 8 carbon atoms and the appropriate number of hydrogen, e.g. an alkyl group. It may be linear or branched. It may contain a cycloalkyl group. For parts of the range C₁₋₆ saturated hydrocarbon group” all subgroups thereof are contemplated, such as C₁₋₇, C₁₋₆, C₁₋₅, C₁₋₄, C₁₋₃, C₁₋₂, C₁, C₂₋₈, C₂₋₇, C₂₋₆, C₂₋₅, C₂₋₄, C₂₋₃, C₂, C₃₋₈, C₃₋₇, C₃₋₆, C₃₋₅, C₃₋₄, C₃, C₄₋₈, C₄₋₇, C₄₋₆, C₄₋₅, C₄, C₅₋₈, C₅₋₇, C₅₋₆, C₅, C₆₋₈, C₆₋₇, C₆, C₇₋₈, C₇, and C₈ saturated hydrocarbon groups. Non-limiting examples of “C₁₋₈ saturated hydrocarbon group” include methyl, ethyl, n-propyl, isopropyl, n-butyl, isobutyl, sec-butyl, t-butyl, linear and branched pentyl, hexyl, septyl, octyl, cyclopropyl, cyclobutyl, cyclopentyl, cyclohexyl, as well as the following groups.

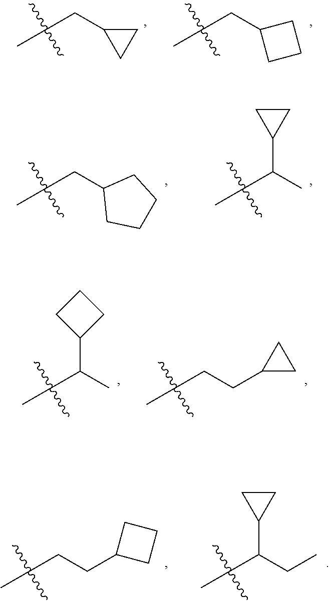

The “C₁₋₈ hydrocarbon group” may be saturated or unsaturated. By unsaturated it is meant that the group contains one or more carbon-carbon double or triple bonds and/or aromatic rings. Examples of unsaturated hydrocarbon groups include ethylenyl, propylenyl, isopropylenyl, butylenyl, and phenyl.

The terms “optional” or “optionally” denotes that the subsequently described event or circumstance may, but need not, occur, and that the description includes instances where the event or circumstance occurs and instances in which it does not.

The C₁₋₈ saturated hydrocarbon group is optionally substituted with 1 to 6 halo.

The term “substituted” denotes that the group to which it refers has one or more hydrogen atoms substituted for a different group. For instance, “substituted hydrocarbon group” refers to a monovalent radical of a hydrocarbon group with one or more hydrogens attached to the hydrocarbon being replaced with another group.

In view of the above, the term “optionally substituted” means that the group to which it refers may or may not be substituted, e.g. for instance, with one or more halo.

As used herein, the term “halo” denotes a halogen atom, and is preferably, F, Cl, Br or I, more preferably F or Cl unless otherwise stated.

A “benzyl group” is

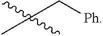 [SMILES: *Cc1ccccc1]

R¹ and R^{1a} may be linked, together with the nitrogen to which they are attached, to form a 3 to 6 membered saturated ring optionally containing an additional heteroatom and optionally substituted with 1 to 6 halo, preferably F. This means that R¹ and R^{1a} are part of a saturated heterocycle that contains 3 to 6 ring atom, i.e. 3, 4, 5 or 6 ring atoms. It is preferrable that this forms a 4 to 6 saturated heterocycle. Non-limiting examples of this heterocycle group include the mono-radicle of the following groups.

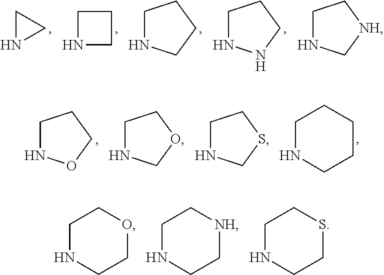

When R¹ and R^{1a} are linked, it is preferred that they form an azetidine, which may optionally be substitute with 1 to 6 halo, preferably F.

As used herein, a “heteroatom” is N, O or S, preferably N, or O.

It is preferable that R^{1a} is H. In this case R¹ is selected from H; a C₁₋₆ saturated hydrocarbon group optionally containing a cycloalkyl group and optionally substituted with 1 to 6 halo; or a benzyl group optionally substituted with 1 to 6 halo. However, it is preferable that R¹ is H or C₁₋₆ saturated hydrocarbon group optionally containing a cycloalkyl group and optionally substituted with 1 to 6 halo, and more preferably H, methyl, ethyl, propyl, or cyclopropyl, each of which is optionally substituted with 1 to 6 halo. Most preferably, R^{1a} is H, and R¹ is H, methyl or cyclopropyl.

Alternatively, when R¹ and R^{1a} are linked, together with the nitrogen to which they are attached, to form a 3-6 membered saturated ring, it is preferrable that the ring formed is an azetidine, pyrrolidine or piperidine, each of which is optionally substituted with 1 to 6 halo.

In formula (I), R² is selected from a C₃₋₆ cycloalkyl, C₁₋₄ alkyl and saturated 3- to 6-membered heterocyclic ring, each of which are optionally substituted with one or more oxo, hydroxy and halo groups. It is highly preferred that the saturated 3- to 6-membered heterocyclic ring does not contain an oxygen atom in the ring. Alternatively, R² may be —(CH₂)ₚCONHR⁶, or —(CH₂)ₚCO₂R⁶. In those two cases, R⁶ is selected from H, C₁₋₃ alkyl, and C₁₋₃ haloalkyl.

In formula (I′), R² is C₃₋₆ cycloalkyl which is optionally substituted with one or more oxo, hydroxy and halo groups.

As used herein, “C₃₋₆ cycloalkyl” denotes a monocyclic alkyl group having 3 to 6 carbon atoms. For parts of the range “C₃₋₆ cycloalkyl” all subgroups thereof are contemplated, such as C₃₋₆, C₃₋₅, C₃₋₄, C₃, C₄₋₆, C₄₋₅, C₄, C₅₋₆, C₅, C₆ cycloalkyl. Examples of these cycloalkyl groups include cyclopropyl, cyclobutyl, cyclopentyl, and cyclohexyl.

The term “saturated 3- to 6-membered heterocyclic ring” is similar to “C₃₋₆ cycloalkyl”, except that one or more of the carbon atoms in that ring have been substituted for a heteroatom, e.g. N or O. For parts of the range “saturated 3-6 membered heterocyclic ring” all subgroups thereof are contemplated, such as 3-6, 3-5, 3-4, 3, 4-6, 4-5, 4, 5-6, 5, and 6 membered heterocyclic ring. Examples of these heterocyclic rings include aziridine, azetidine, pyrrolidine, piperidine, tetrahydrofuran, tetrahydrothiophene, piperazine, tetrahydropyran, thiane, and morpholine.

The term “oxo” is ═O.

The term “C₁₋₃ haloalkyl” is a C₁₋₃ saturated hydrocarbon group as defined above, i.e. an alkyl, in which one or more of the hydrogen atoms is replaced with halo, e.g. F, Cl, Br, or I, preferably F or Cl, more preferably F. Each halo-substituted carbon atom in C₁₋₃ haloalkyl may be mono-, di- or, where possible, trisubstituted with an independently selected halo atom. For parts of the range “C₁₋₃ haloalkyl” all subgroups thereof are contemplated, such as C₁₋₃, C₁₋₂, C₁, C₂₋₃, C₂, and C₃ haloalkyl. Non-limiting example of C₁₋₃ haloalkyl include —CH₂F, —CF₂H, —CF₃, —CH₂Cl, —CCl₂H, —CCl₃, —CHFCl, —CF₂Cl, —CCl₂F, —CH₂CF₃, —CF₂CF₃, —CF₂CF₂CF₃, and —CH₂CH₂CF₃.

In the compounds of Formula (I), it is preferrable that R² is selected from C₃₋₆ cycloalkyl, C₁₋₄ alkyl, and a saturated 5- or 6-membered heterocyclic ring (preferably a saturated 5- or 6-membered heterocyclic ring that does not contain an oxygen atom in the ring), optionally substituted with one or more oxo, hydroxy and halo groups. The C₃₋₆ cycloalkyl and C₁₋₄ alkyl groups or heterocyclic ring may in some instances be substituted by one to three halo, and/or one oxo.

In particular, it is preferable that R² is

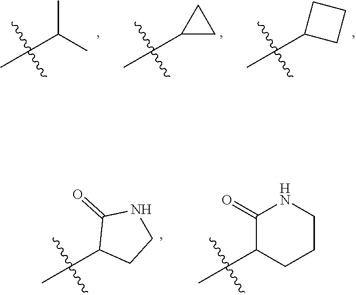

each of which is optionally substituted by one or more halo, preferably F.

It is more preferrable that R² is cyclic in nature. Therefore, it is more preferrable that R² is selected from a C₃₋₆ cycloalkyl and saturated 3- to 6-membered heterocyclic ring (preferably a saturated 3- or 6-membered heterocyclic ring that does not contain an oxygen atom in the ring), for instance a C₃₋₆ cycloalkyl and a saturated 5- or 6-membered heterocyclic ring (preferably a saturated 5- or 6-membered heterocyclic ring that does not contain an oxygen atom in the ring), each of which are optionally substituted with one or more oxo, hydroxy and halo groups. In this case, the C₃₋₆ cycloalkyl group and heterocyclic ring may in some instances also be substituted by one to three halo, and/or one oxo.

In view of this, R² is most preferably

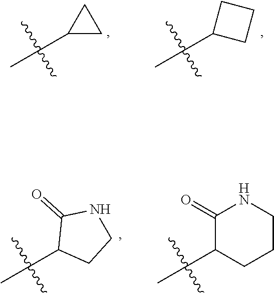

each of which is optionally substituted by one or more halo, preferably F.

In Formula (I′), R² is most preferably

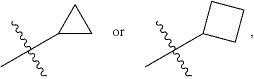

each of which is optionally substituted by one or more halo, preferably F.

In the compounds described herein, R represents one of two sub-groups. There are

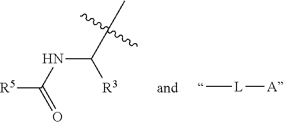

When R is the former of the two group, then the compounds of Formula (I) or (I′) are a compound of Formula (IA)

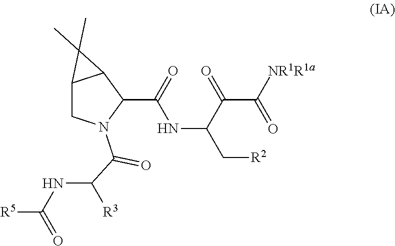

or a salt, solvate, hydrate, N-oxide or prodrug thereof,
wherein R¹, R^{1a}, R², R³ and R⁵ are as herein defined.

The compounds of formula (IA) above may optionally be compounds according to formula (IAi) below:

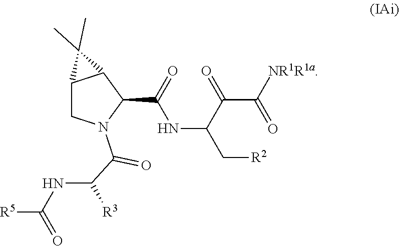

wherein the stereochemistry is as shown above, and the substituents are as defined elsewhere herein.

When R is -L-A, then the compounds of Formula (I) or (I′) are a compound of Formula (IB)

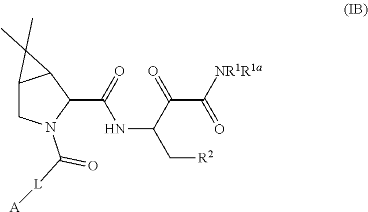

or a salt, solvate, hydrate, N-oxide or prodrug thereof,
wherein R¹, R^{1a}, L and A are as herein defined.

In the compounds of Formula (IA), R³ is selected from
(i) a saturated group containing 1 to 6 carbon atoms and optionally containing a cycloalkyl group; or
(ii) a saturated ring containing an oxygen or nitrogen heteroatom,
said saturated group or ring being optionally substituted with one or more substituents chosen from fluorine, hydroxy or methoxy.

Alternatively, R³ is —CH₂aryl; —CH(CH₃)aryl; or —C(CH₃)₂aryl.

The term “saturated group containing 1 to 6 carbon atoms” is a C₁₋₆ saturated hydrocarbon group as defined above, and may optionally contain a cycloalkyl group as part of those 1 to 6 carbon atoms.

The saturated ring containing oxygen or nitrogen is a 3- to 6-membered saturated heterocyclic ring as defined above.

Preferred group for R³ include methyl, ethyl, propyl, (especially iso-propyl), tert-butyl, sec-butyl, iso-butyl, as well as the following groups.

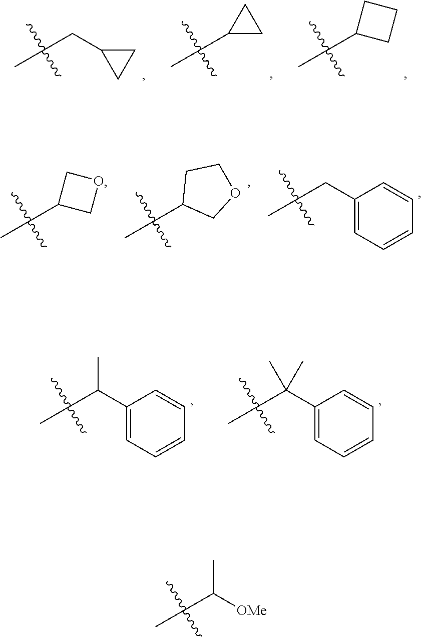

More preferred groups for R³ are

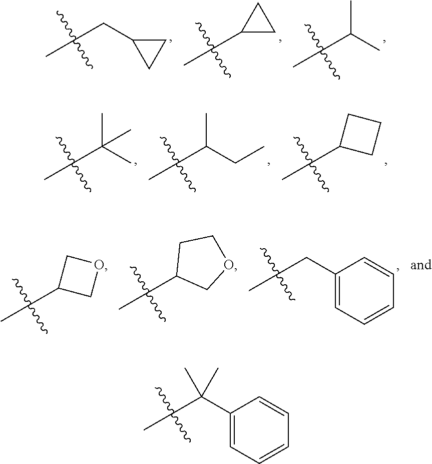

each of which are optionally substituted with one or more halo, preferably F

The more preferred groups for R³ are methyl, iso-propyl, tert-butyl and

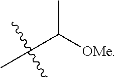 [SMILES: *C(C)OC]

Most preferably, R³ is iso-propyl or tert-butyl.

R⁵ is a (saturated or unsaturated) C₁₋₈ hydrocarbon group, optionally containing one or more rings or a double bond and which is optionally substituted with one or more groups selected from halo; cyano; hydroxy; methoxy; amino; and a cycloalkyl, heterocycloalkyl, aryl or heteroaryl. Preferably R⁵ is a saturated or unsaturated C₁₋₅ hydrocarbon group, optionally substituted with one or more halo, preferably F.

The term “aryl” as used herein is a C aromatic group, also known as phenyl.

The term “heteroaryl” is a 5- or 6-membered aromatic ring that contains at least one heteroatom. Suitable heteroatoms include O, N and S, preferably O or N. Non-limiting examples of heteroaryl groups include pyrrole, pyrazole, imidazole, triazole, tetrazole, furan, thiophene, oxazole, isoxazole, isothiazole, thiazole, pyridine, pyridazine, pyrimidine, pyrazine, and triazine.

Preferred examples of R⁵ include methyl, —CF₃, ethyl, propyl, (especially iso-propyl), cyclopropyl, butyl, and cyclobutyl, and more preferably —CF₃, iso-propyl cyclopropyl,

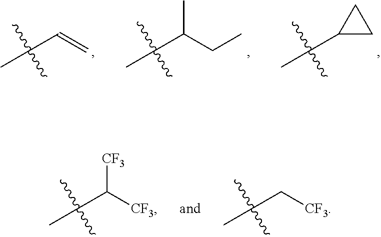

More preferably, R⁵ is

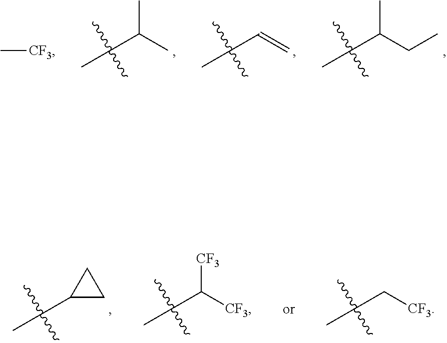

Most preferably, R⁵ is —CF₃ or

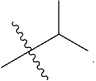 [SMILES: *C(C)C]

As mentioned above, R may be -L-A, in which case the compounds of Formula (I) or (I′) are a compound of Formula (IB).

In -L-A, group L is a linker, i.e. it is divalent, and is selected from —CR⁷═CR⁸—, —CHR⁹—CHR¹⁰— or —O—CHR¹¹—.

R⁷ to R¹¹ (i.e. R⁷, R⁸, R⁹, R¹⁰, and R¹¹) are independently H, —(CH₂)ₘCO₂R¹² or C₁₋₃ alkyl, wherein the C₁₋₃ alkyl is optionally substituted with 1 to 6 (i.e. 1, 2, 3, 4, 5, or 6) fluorine atoms. m is independently 0 to 3. R¹² is selected from H, C₁₋₃ alkyl, and C₁₋₃ haloalkyl.

In a particular case, group R⁹ and R¹⁰ are joined to form a cyclopropyl. In this case, L is

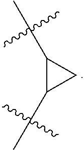 [SMILES: *C1CC1*]

It is preferred that L is —CH═CH—, —CH₂CH₂—, —CH₂—CH(CH₂CH₃)—, —CH₂—CH(CH₂CO₂H)—, —OCH₂—, —CH₂—CH(CH₃)—, —CH₂—CH(CF₃)—, —OCH(CH₃)—, or

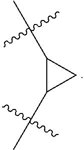 [SMILES: *C1CC1*]

Most preferably, L is —CH═CH—.

Group ‘A’ is a phenyl or heteroaryl group (preferably 6-membered heteroaryl group). Those two groups are optionally substituted with one or more groups selected from halo, —CN, —CO₂R¹³, —OR¹³, —SO₂R¹³, —SONHR¹³, —OSO₂R¹³, —PO(R¹³)₂, —SF₅ and C₁₋₃ alkyl. Each R¹³ is independently selected from H, C₁₋₃ alkyl, and C₁₋₃ haloalkyl.

Group ‘A’ is preferably phenyl or pyridinyl optionally substituted with one or more groups selected from halo, hydroxy and C₁₋₃ alkyl. More preferably, group ‘A’ is phenyl or pyridinyl optionally substituted with one or more halo (particularly F and Cl). Particular ‘A’ groups of interest are

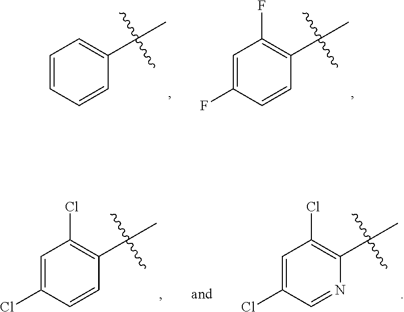 [SMILES: *c1ccccc1 ; *c1ccc(F)cc1F ; *c1ccc(Cl)cc1Cl ; *c1ncc(Cl)cc1Cl]

In view of the above, preferred groups for “-L-A” are

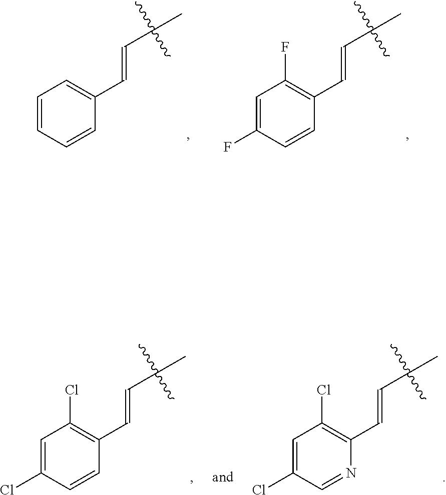 [SMILES: */C=C/c1ccccc1 ; */C=C/c1ccc(F)cc1F ; */C=C/c1ccc(Cl)cc1Cl ; */C=C/c1ncc(Cl)cc1Cl]

Notwithstanding the above, when R² is a C₃₋₅ saturated hydrocarbon group containing a cycloalkyl group which is unsubstituted or substituted with one or more substituents chosen from fluorine or hydroxyl, then R represents

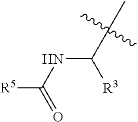

in which R⁵ is methyl, optionally substituted with one or more substituents chosen from halo; cyano; hydroxy; methoxy; amino; with R³ as defined herein; or R represents -L-A as defined herein.

The compounds of Formula (I) may be compounds according to formula (IIA)

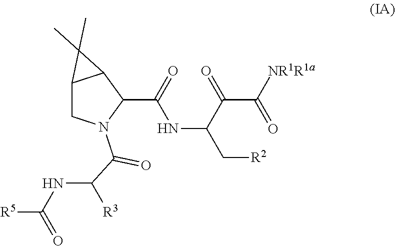

or a salt, solvate, hydrate, N-oxide or prodrug thereof
   wherein
   R^{1a} is H;
   R¹ is H, methyl, ethyl, propyl, or cyclopropyl, each of which is optionally substituted with 1 to 6 halo. preferably F; or
   R¹ and R^{1a} are linked, together with the nitrogen to which they are attached, to form an azetidine, pyrrolidine or piperidine ring, which is optionally substituted with 1 to 6 halo, preferably F;
   R² is C₃₋₆ cycloalkyl, C₁₋₄ alkyl or a 5- or 6-membered heterocyclic ring (preferably a saturated 5- or 6-membered heterocyclic ring that does not contain an oxygen atom in the ring), optionally substituted with one or more oxo, hydroxy and halo groups;
   R represents:

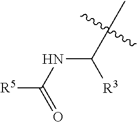

wherein R³ is selected from methyl, ethyl, propyl, iso-propyl, tert-butyl, sec-butyl, iso-butyl, as well as the following groups

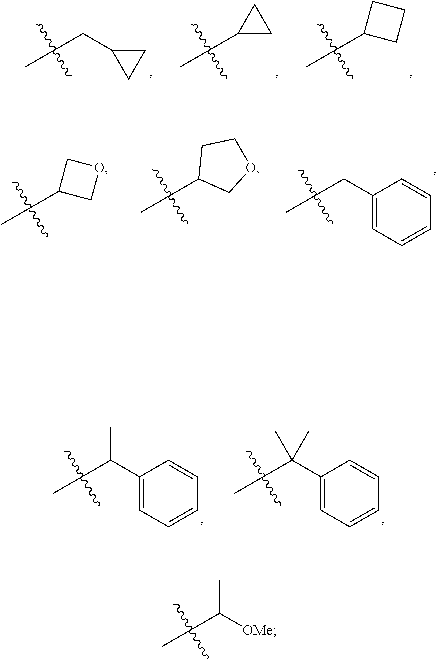

and
R⁵ is a saturated or unsaturated C₁₋₅ hydrocarbon group, optionally substituted with one or more halo, preferably F; or
R represents -L-A
   wherein L is —CH═CH—, —CH₂CH₂—, —CH₂—CH(CH₂CH₃)—, —CH₂—CH(CH₂CO₂H)—, —OCH₂—, —CH₂—CH(CH₃)—, —CH₂—CH(CF₃)—, —OCH(CH₃)—, or

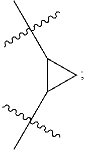 [SMILES: *C1CC1*]

and
A is phenyl or pyridinyl optionally substituted with one or more groups selected from halo (preferably fluoro), hydroxy and C₁₋₃ alkyl.

In addition, the compounds of the invention are preferably not:
(1R,2S,5S)—N-(4-Amino-1-cyclopropyl-3,4-dioxobutan-2-yl)-3-((S)-3,3-dimethyl-2-(2,2,2-trifluoroacetamido)butanoyl)-6,6-dimethyl-3-azabicyclo[3.1.0]hexane-2-carboxamide; or
(1R,2S,5S)—N-(4-(Azetidin-1-yl)-1-cyclopropyl-3,4-dioxobutan-2-yl)-3-((S)-3,3-dimethyl-2-(2,2,2-trifluoroacetamido)butanoyl)-6,6-dimethyl-3-azabicyclo[3.1.0]hexane-2-carboxamide.

Preferably, the present invention does not include salts of said compounds. Still more preferably the present invention does not include salts, solvates, hydrates, N-oxides or prodrugs of said compounds.

Preferably, the present invention does not include compounds of the following structures:

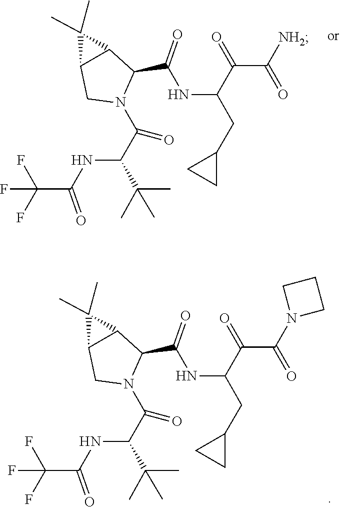

Preferred compounds described herein are selected from
N-(4-(cyclopropylamino)-3,4-dioxo-1-(2-oxopyrrolidin-3-yl)butan-2-yl)-3-(3,3-dimethyl-2-(2,2,2-trifluoroacetamido)butanoyl)-6,6-dimethyl-3-azabicyclo[3.1.0]hexane-2-carboxamide;
N-[1-(cyclopropylmethyl)-3-(methylamino)-2,3-dioxo-propyl]-3-[3,3-dimethyl-2-[(2,2,2-trifluoroacetyl)amino]butanoyl]-6,6-dimethyl-3-azabicyclo[3.1.0]hexane-2-carboxamide;
N-[3-amino-1-(cyclopropylmethyl)-2,3-dioxo-propyl]-6,6-dimethyl-3-[3-methyl-2-[(2,2,2-trifluoroacetyl)amino]butanoyl]-3-azabicyclo[3.1.0]hexane-2-carboxamide:
N-[3-amino-1-(cyclobutylmethyl)-2,3-dioxo-propyl]-3-[3,3-dimethyl-2-[(2,2,2-trifluoroacetyl)amino]butanoyl]-6,6-dimethyl-3-azabicyclo[3.1.0]hexane-2-carboxamide;
N-[3-(cyclopropylamino)-2,3-dioxo-1-[[2-oxopyrrolidin-3-yl]methyl]propyl]-3-[3,3-dimethyl-2-(2-methylpropanoylamino)butanoyl]-6,6-dimethyl-3-azabicyclo[3.1.0]hexane-2-carboxamide;
N-[3-(cyclopropylamino)-2,3-dioxo-1-[[2-oxo-3-piperidyl]methyl]propyl]-3-[3,3-dimethyl-2-[(2,2,2-trifluoroacetyl)amino]butanoyl]-6,6-dimethyl-3-azabicyclo[3.1.0]hexane-2-carboxamide; and
N-(4-(cyclopropylamino)-3,4-dioxo-1-(2-oxopyrrolidin-3-yl)butan-2-yl)-3-(3-(2,4-difluorophenyl)acryloyl)-6,6-dimethyl-3-azabicyclo[3.1.0]hexane-2-carboxamide,
   or salt, solvate, hydrate, N-oxide or prodrug thereof.

Further preferred compounds described herein are selected from
(1R,2S,5S)—N-(4-(cyclopropylamino)-3,4-dioxo-1-((S)-2-oxopyrrolidin-3-yl)butan-2-yl)-3-((S)-3,3-dimethyl-2-(2,2,2-trifluoroacetamido)butanoyl)-6,6-dimethyl-3-azabicyclo[3.1.0]hexane-2-carboxamide;
(1R,2S,5S)—N-[1-(cyclopropylmethyl)-3-(methylamino)-2,3-dioxo-propyl]-3-[(2S)-3,3-dimethyl-2-[(2,2,2-trifluoroacetyl)amino]butanoyl]-6,6-dimethyl-3-azabicyclo[3.1.0]hexane-2-carboxamide;
(1R,2S,5S)—N-[3-amino-1-(cyclopropylmethyl)-2,3-dioxo-propyl]-6,6-dimethyl-3-[(2S)-3-methyl-2-[(2,2,2-trifluoroacetyl)amino]butanoyl]-3-azabicyclo[3.1.0]hexane-2-carboxamide;
(1R,2S,5S)—N-[3-amino-1-(cyclobutylmethyl)-2,3-dioxo-propyl]-3-[(2S)-3,3-dimethyl-2-[(2,2,2-trifluoroacetyl)amino]butanoyl]-6,6-dimethyl-3-azabicyclo[3.1.0]hexane-2-carboxamide;
(1R,2S,5S)—N-[3-(cyclopropylamino)-2,3-dioxo-1-[[(3S)-2-oxopyrrolidin-3-yl]methyl]propyl]-3-[(2S)-3,3-dimethyl-2-(2-methylpropanoylamino)butanoyl]-6,6-dimethyl-3-azabicyclo[3.1.0]hexane-2-carboxamide;
(1R,2S,5S)—N-[3-(cyclopropylamino)-2,3-dioxo-1-[[(3R)-2-oxo-3-piperidyl]methyl]propyl]-3-[(2S)-3,3-dimethyl-2-[(2,2,2-trifluoroacetyl)amino]butanoyl]-6,6-dimethyl-3-azabicyclo[3.1.0]hexane-2-carboxamide; and
(1R,2S,5S)—N-(4-(cyclopropylamino)-3,4-dioxo-1-((S)-2-oxopyrrolidin-3-yl)butan-2-yl)-3-((E)-3-(2,4-difluorophenyl)acryloyl)-6,6-dimethyl-3-azabicyclo[3.1.0]hexane-2-carboxamide,
   or salt, solvate, hydrate, N-oxide or prodrug thereof.

More preferred compounds described herein may be selected from
N-[1-(cyclopropylmethyl)-3-(methylamino)-2,3-dioxo-propyl]-3-[3,3-dimethyl-2-[(2,2,2-trifluoroacetyl)amino]butanoyl]-6,6-dimethyl-3-azabicyclo[3.1.0]hexane-2-carboxamide;
N-[3-amino-1-(cyclopropylmethyl)-2,3-dioxo-propyl]-6,6-dimethyl-3-[3-methyl-2-[(2,2,2-trifluoroacetyl)amino]butanoyl]-3-azabicyclo[3.1.0]hexane-2-carboxamide;
N-[3-amino-1-(cyclobutylmethyl)-2,3-dioxo-propyl]-3-[3,3-dimethyl-2-[(2,2,2-trifluoroacetyl)amino]butanoyl]-6,6-dimethyl-3-azabicyclo[3.1.0]hexane-2-carboxamide;
N-[3-(cyclopropylamino)-2,3-dioxo-1-[[2-oxopyrrolidin-3-yl]methyl]propyl]-3-[3,3-dimethyl-2-(2-methylpropanoylamino)butanoyl]-6,6-dimethyl-3-azabicyclo[3.1.0]hexane-2-carboxamide;
N-[3-(cyclopropylamino)-2,3-dioxo-1-[[2-oxo-3-piperidyl]methyl]propyl]-3-[3,3-dimethyl-2-[(2,2,2-trifluoroacetyl)amino]butanoyl]-6,6-dimethyl-3-azabicyclo[3.1.0]hexane-2-carboxamide; and
N-(4-(cyclopropylamino)-3,4-dioxo-1-(2-oxopyrrolidin-3-yl)butan-2-yl)-3-(3-(2,4-difluorophenyl)acryloyl)-6,6-dimethyl-3-azabicyclo[3.1.0]hexane-2-carboxamide
   or salt, solvate, hydrate, N-oxide or prodrug thereof.

Further preferred compounds may be selected from
(1R,2S,5S)—N-[1-(cyclopropylmethyl)-3-(methylamino)-2,3-dioxo-propyl]-3-[(2S)-3,3-dimethyl-2-[(2,2,2-trifluoroacetyl)amino]butanoyl]-6,6-dimethyl-3-azabicyclo[3.1.0]hexane-2-carboxamide:
(1R,2S,5S)—N-[3-amino-1-(cyclopropylmethyl)-2,3-dioxo-propyl]-6,6-dimethyl-3-[(2S)-3-methyl-2-[(2,2,2-trifluoroacetyl)amino]butanoyl]-3-azabicyclo[3.1.0]hexane-2-carboxamide;
(1R,2S,5S)—N-[3-amino-1-(cyclobutylmethyl)-2,3-dioxo-propyl]-3-[(2S)-3,3-dimethyl-2-[(2,2,2-trifluoroacetyl)amino]butanoyl]-6,6-dimethyl-3-azabicyclo[3.1.0]hexane-2-carboxamide;
(1R,2S,5S)—N-[3-(cyclopropylamino)-2,3-dioxo-1-[[(3S)-2-oxopyrrolidin-3-yl]methyl]propyl]-3-[(2S)-3,3-dimethyl-2-(2-methylpropanoylamino)butanoyl]-6,6-dimethyl-3-azabicyclo[3.1.0]hexane-2-carboxamide;
(1R,2S,5S)—N-[3-(cyclopropylamino)-2,3-dioxo-1-[[(3R)-2-oxo-3-piperidyl]methyl]propyl]-3-[(2S)-3,3-dimethyl-2-[(2,2,2-trifluoroacetyl)amino]butanoyl]-6,6-dimethyl-3-azabicyclo[3.1.0]hexane-2-carboxamide; and
(1R,2S,5S)—N-(4-(cyclopropylamino)-3,4-dioxo-1-((S)-2-oxopyrrolidin-3-yl)butan-2-yl)-3-((E)-3-(2,4-difluorophenyl)acryloyl)-6,6-dimethyl-3-azabicyclo[3.1.0]hexane-2-carboxamide,
   or salt, solvate, hydrate, N-oxide or prodrug thereof.

More preferred compounds may be selected from:
N-[1-(cyclopropylmethyl)-3-(methylamino)-2,3-dioxo-propyl]-3-[3,3-dimethyl-2-[(2,2,2-trifluoroacetyl)amino]butanoyl]-6,6-dimethyl-3-azabicyclo[3.1.0]hexane-2-carboxamide;
N-[3-amino-1-(cyclopropylmethyl)-2,3-dioxo-propyl]-6,6-dimethyl-3-[3-methyl-2-[(2,2,2-trifluoroacetyl)amino]butanoyl]-3-azabicyclo[3.1.0]hexane-2-carboxamide;
N-[3-amino-1-(cyclobutylmethyl)-2,3-dioxo-propyl]-3-[3,3-dimethyl-2-[(2,2,2-trifluoroacetyl)amino]butanoyl]-6,6-dimethyl-3-azabicyclo[3.1.0]hexane-2-carboxamide;
   or salt, solvate, hydrate, N-oxide or prodrug thereof.

Even more preferred compounds may be selected from:
(1R,2S,5S)—N-[1-(cyclopropylmethyl)-3-(methylamino)-2,3-dioxo-propyl]-3-[(2S)-3,3-dimethyl-2-[(2,2,2-trifluoroacetyl)amino]butanoyl]-6,6-dimethyl-3-azabicyclo[3.1.0]hexane-2-carboxamide;
(1R,2S,5S)—N-[3-amino-1-(cyclopropylmethyl)-2,3-dioxo-propyl]-6,6-dimethyl-3-[(2S)-3-methyl-2-[(2,2,2-trifluoroacetyl)amino]butanoyl]-3-azabicyclo[3.1.0]hexane-2-carboxamide;
(1R,2S,5S)—N-[3-amino-1-(cyclobutylmethyl)-2,3-dioxo-propyl]-3-[(2S)-3,3-dimethyl-2-[(2,2,2-trifluoroacetyl)amino]butanoyl]-6,6-dimethyl-3-azabicyclo[3.1.0]hexane-2-carboxamide;
   or salt, solvate, hydrate, N-oxide or prodrug thereof.

The invention will now be illustrated, but not limited, by reference to the following examples shown in Table 1.

TABLE 1
Non-limiting compounds

| | | |
|---|---|---|
| Example 1 | 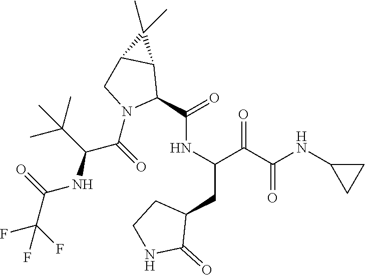 [SMILES: CC(C)(C)[C@H](NC(=O)C(F)(F)F)C(=O)N1C[C@H]2[C@@H]([C@H]1C(=O)NC(C[C@@H]1CCNC1=O)C(=O)C(=O)NC1CC1)C2(C)C] | (1R,2S,5S)-N-(4-(cyclopropylamino)-3,4- dioxo-1-((S)-2-oxopyrrolidin-3-yl)butan-2- yl)-3-((S)-3,3-dimethyl-2-(2,2,2- trifluoroacetamido)butanoyl)-6,6- dimethyl-3-azabicyclo[3.1.0]hexane-2- carboxamide |
| | Chiral | |
| Example 2 | 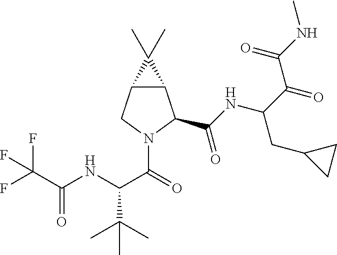 [SMILES: CNC(=O)C(=O)C(CC1CC1)NC(=O)[C@@H]1[C@@H]2[C@H](CN1C(=O)[C@@H](NC(=O)C(F)(F)F)C(C)(C)C)C2(C)C] | (1R,2S,5S)-N-[1-(cyclopropylmethyl)-3- (methylamino)-2,3-dioxo-propyl]-3-[(2S)- 3,3-dimethyl-2-[(2,2,2- trifluoroacetyl)amino]butanoyl]-6,6- dimethyl-3-azabicyclo[3.1.0]hexane-2- carboxamide |
| | Chiral | |
| Example 3 | 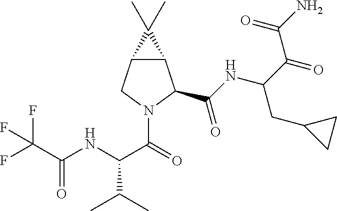 [SMILES: CC(C)[C@H](NC(=O)C(F)(F)F)C(=O)N1C[C@H]2[C@@H]([C@H]1C(=O)NC(CC1CC1)C(=O)C(N)=O)C2(C)C] | (1R,2S,5S)-N-[3-amino-1- (cyclopropylmethyl)-2,3-dioxo-propyl]- 6,6-dimethyl-3-[(2S)-3-methyl-2-[(2,2,2- trifluoroacetyl)amino]butanoyl]-3- azabicyclo[3.1.0]hexane-2-carboxamide |
| | Chiral | |
| Example 4 | 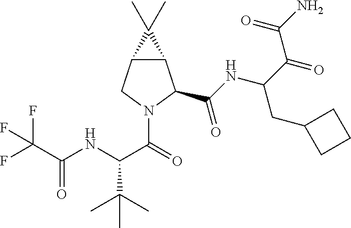 [SMILES: CC(C)(C)[C@H](NC(=O)C(F)(F)F)C(=O)N1C[C@H]2[C@@H]([C@H]1C(=O)NC(CC1CCC1)C(=O)C(N)=O)C2(C)C] | (1R,2S,5S)-N-[3-amino-1- (cyclobutylmethyl)-2,3-dioxo-propyl]-3- [(2S)-3,3-dimethyl-2-[(2,2,2- trifluoroacetyl)amino]butanoyl]-6,6- dimethyl-3-azabicyclo[3.1.0]hexane-2- carboxamide |
| | Chiral | |
| Example 5 | 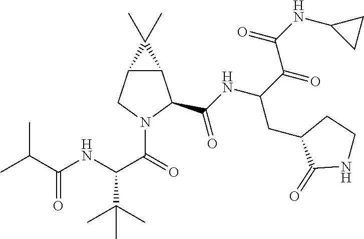 [SMILES: CC(C)C(=O)N[C@H](C(=O)N1C[C@H]2[C@@H]([C@H]1C(=O)NC(C[C@@H]1CCNC1=O)C(=O)C(=O)NC1CC1)C2(C)C)C(C)(C)C] | (1R,2S,5S)-N-[3-(cyclopropylamino)-2,3- dioxo-1-[(3S)-2-oxopyrrolidin-3- yl]methyl]propyl]-3-[(2S)-3,3-dimethyl-2- (2-methylpropanoylamino)butanoyl]-6,6- dimethyl-3-azabicyclo[3.1.0]hexane-2- carboxamide |
| | Chiral | |
| Example 6 | 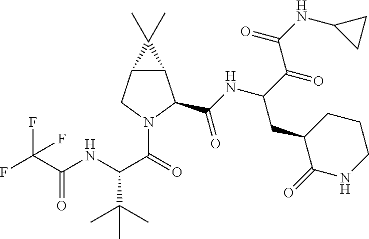 [SMILES: CC(C)(C)[C@H](NC(=O)C(F)(F)F)C(=O)N1C[C@H]2[C@@H]([C@H]1C(=O)NC(C[C@H]1CCCNC1=O)C(=O)C(=O)NC1CC1)C2(C)C] | (1R,2S,5S)-N-[3-(cyclopropylamino)-2,3- dioxo-1-[[(3R)-2-oxo-3- piperidyl]methyl]propyl]-3-[(2S)-3,3- dimethyl-2-[(2,2,2- trifluoroacetyl)amino]butanoyl]-6,6- dimethyl-3-azabicyclo[3.1.0]hexane-2- carboxamide |
| | Chiral | |
| Example 7 | 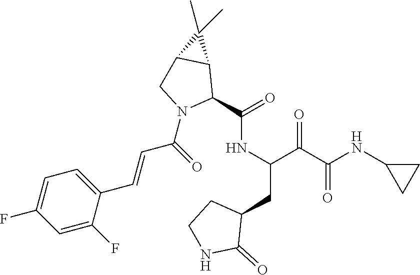 [SMILES: CC1(C)[C@@H]2[C@@H](C(=O)NC(C[C@@H]3CCNC3=O)C(=O)C(=O)NC3CC3)N(C(=O)/C=C/c3ccc(F)cc3F)C[C@@H]21] | (1R,2S,5S)-N-(4-(cyclopropylamino)-3,4- dioxo-1-((S)-2-oxopyrrolidin-3-yl)butan-2- yl)-3-((E)-3-(2,4-difluorophenyl)acryloyl)- 6,6-dimethyl-3-azabicyclo[3.1.0]hexane- 2-carboxamide |
| | Chiral | |

Examples falling outside the scope of the claims are intended for reference purposes.

In this disclosure, where a discrepancy exists between a compound name and a compound structure, the compound structure will prevail.

The compounds of the invention may be used as such or, where appropriate, as salts, in particular pharmacologically acceptable salts (acid or base addition salts), thereof. The pharmacologically acceptable addition salts mentioned below are meant to comprise the therapeutically active non-toxic acid and base addition salt forms that the compounds are able to form. Compounds that have basic properties can be converted to their pharmaceutically acceptable acid addition salts by treating the base form with an appropriate acid. Exemplary acids include inorganic acids, such as hydrogen chloride, hydrogen bromide, hydrogen iodide, sulphuric acid, phosphoric acid; and organic acids such as formic acid, acetic acid, propanoic acid, hydroxyacetic acid, lactic acid, pyruvic acid, glycolic acid, maleic acid, malonic acid, oxalic acid, benzenesulphonic acid, toluenesulphonic acid, methanesulphonic acid, trifluoroacetic acid, fumaric acid, succinic acid, malic acid, tartaric acid, citric acid, salicylic acid, p-aminosalicylic acid, pamoic acid, benzoic acid, ascorbic acid and the like. Exemplary base addition salt forms are the sodium, potassium, calcium salts, and salts with pharmaceutically acceptable amines such as, for example, ammonia, alkylamines, benzathine, and amino acids, such as, e.g. arginine and lysine. The term addition salt as used herein also comprises solvates which the compounds and salts thereof are able to form, such as, for example, hydrates, alcoholates and the like.

Throughout the present disclosure, a given chemical formula or name shall also encompass all (pharmaceutically acceptable) salts, solvates, hydrates, N-oxides, and/or prodrug forms thereof. It is to be understood that the compounds of the invention include any and all hydrates and/or solvates of the compound formulae.

It is appreciated that certain functional groups, such as the hydroxy, amino, and like groups form complexes and/or coordination compounds with water and/or various solvents, in the various physical forms of the compounds. Accordingly, the above formulas are to be understood to include and represent those various hydrates and/or solvates.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H, 2H- and 4H-1,2,4-triazole, 1H- and 2H-isoindole, and 1H- and 2H-pyrazole. Tautomeric forms can be in equilibrium or sterically locked into one form by appropriate substitution.

The compounds described herein can be asymmetric (e.g. having one or more stereocenters). All stereoisomers, such as enantiomers and diastereoisomers, are intended unless otherwise indicated. The terms “diastereoisomer” and “diastereomer” are used herein interchangeably. Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis- and trans-geometric isomers of the compounds of the present invention are described and may be isolated as a mixture of isomers or as separated isomeric forms.

In the case of the compounds which contain an asymmetric carbon atom, the invention relates to the D form, the L form, and D,L mixtures and also, where more than one asymmetric carbon atom is present, to the diastereoisomeric forms.

Those compounds of the invention which contain asymmetric carbon atoms, and which as a rule accrue as racemates, can be separated into the optically active isomers in a known manner, for example using an optically active acid. However, it is also possible to use an optically active starting substance from the outset, with a corresponding optically active or diastereoisomeric compound then being obtained as the end product.

The compounds of the invention may comprise isotopic form of the atoms therein, e.g. they may be isotopically-labelled and/or isotopically-enriched forms of the compounds. The compounds of the invention herein may contain unnatural proportions of atomic isotopes at one or more of the atoms that constitute such compounds. Examples of isotopes that can be incorporated into the disclosed compounds include isotopes of hydrogen, carbon, nitrogen, oxygen, phosphorus, sulfur, chlorine, such as ²H, ³H, ¹¹C, ¹³C, ¹⁴C, ¹³N, ¹⁵O, ¹⁷O, ³²P, ³⁵S, ¹⁸F, ³⁶Cl.

The term “N-oxide” denotes a compound containing the N⁺—O⁻ functional group, such as in the following example.

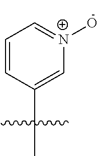 [SMILES: *c1ccc[n+]([O-])c1]

The term “prodrugs” refers to compounds that may be converted under physiological conditions or by solvolysis to a biologically active compound of the invention. A prodrug may be inactive when administered to a subject in need thereof, but is converted in vivo to an active compound of the invention. Prodrugs are typically rapidly transformed in vivo to yield the parent compound of the invention, e.g. by hydrolysis in the blood. The prodrug compound usually offers advantages of solubility, tissue compatibility or delayed release in a mammalian organism (see Silverman, R. B., The Organic Chemistry of Drug Design and Drug Action, 2nd Ed., Elsevier Academic Press (2004), page 498 to 549). Prodrugs of a compound of the invention may be prepared by modifying functional groups, such as a hydroxy, amino or mercapto groups, present in a compound of the invention in such a way that the modifications are cleaved, either in routine manipulation or in vivo, to the parent compound of the invention. Examples of prodrugs include, but are not limited to, acetate, formate and succinate derivatives of hydroxy functional groups or phenyl carbamate derivatives of amino functional groups.

For clinical use, the compounds disclosed herein are formulated into pharmaceutical compositions (or formulations) for various modes of administration. It will be appreciated that compounds of the invention may be administered together with a physiologically acceptable carrier, excipient, and/or diluent (i.e. one, two, or all three of these). The pharmaceutical compositions disclosed herein may be administered by any suitable route, preferably by oral, rectal, nasal, topical (including buccal and sublingual), sublingual, transdermal, intrathecal, transmucosal or parenteral (including subcutaneous, intramuscular, intravenous and intradermal) administration. Other formulations may conveniently be presented in unit dosage form, e.g., tablets and sustained release capsules, and in liposomes, and may be prepared by any methods well known in the art of pharmacy. Pharmaceutical formulations are usually prepared by mixing the active substance, or a pharmaceutically acceptable salt thereof, with conventional pharmaceutically acceptable carriers, diluents or excipients. Examples of excipients are water, gelatin, gum arabicum, lactose, microcrystalline cellulose, starch, sodium starch glycolate, calcium hydrogen phosphate, magnesium stearate, talcum, colloidal silicon dioxide, and the like. Such formulations may also contain other pharmacologically active agents, and conventional additives, such as stabilizers, wetting agents, emulsifiers, flavouring agents, buffers, and the like. Usually, the amount of active compounds is between 0.1-95% by weight of the preparation, preferably between 0.2-20% by weight in preparations for parenteral use and more preferably between 1-50% by weight in preparations for oral administration. The formulations can be further prepared by known methods such as granulation, compression, microencapsulation, spray coating, etc. The formulations may be prepared by conventional methods in the dosage form of tablets, capsules, granules, powders, syrups, suspensions, suppositories or injections. Liquid formulations may be prepared by dissolving or suspending the active substance in water or other suitable vehicles. Tablets and granules may be coated in a conventional manner. To maintain therapeutically effective plasma concentrations for extended periods of time, compounds disclosed herein may be incorporated into slow-release formulations.

In addition to the above, pharmaceutically acceptable excipients can be selected from, for example, carriers (e.g. a solid, liquid or semi-solid carrier), adjuvants, diluents (e.g. solid diluents such as fillers or bulking agents; and liquid diluents such as solvents and co-solvents), granulating agents, binders, flow aids, coating agents, release-controlling agents (e.g. release retarding or delaying polymers or waxes), binding agents, disintegrants, buffering agents, lubricants, preservatives, anti-fungal and antibacterial agents, antioxidants, buffering agents, tonicity-adjusting agents, thickening agents, flavouring agents, sweeteners, pigments, plasticizers, taste masking agents, stabilisers or any other excipients conventionally used in pharmaceutical compositions.

In view of the above, a second aspect of the invention provides a pharmaceutical composition comprising a compound of the invention, including a salt, solvate, hydrate, N-oxide or prodrug thereof, and a pharmaceutically acceptable excipient.

Said pharmaceutical compositions may further comprise one or more drugs which block cypP450 mediated metabolism. Said drugs may be selected from ritonavir, lopinavir or a combination thereof.

The dose level and frequency of dosage of the specific compound will vary depending on a variety of factors including the potency of the specific compound employed, the metabolic stability and length of action of that compound, the patient's age, body weight, general health, sex, diet, mode and time of administration, rate of excretion, drug combination, the severity of the condition to be treated, and the patient undergoing therapy. The daily dosage may, for example, range from about 0.001 mg to about 100 mg per kilo of body weight, administered singly or multiply in doses, e.g. from about 0.01 mg to about 25 mg each. Normally, such a dosage is given orally but parenteral administration may also be chosen.

An object of the present invention relates to the compounds of the invention for use as a medicament. The term ‘medicament’ denotes a substance used for medical treatment or as a medicine. The compounds of the invention may be useful as inhibitors of SARS-CoV-2 Mpro. As such, they are useful in the treatment or prevention of medical conditions (disease or disorder) that are affected by SARS-CoV-2 Mpro.

In view of the above, a third aspect of the invention provides a compound of the invention, or a pharmaceutical composition comprising said compound, for use in the treatment of SARS-CoV-2 or in the treatment of disorders associated with SARS-CoV-2.

In a fourth aspect of the invention, there is provided a use of a compound of the invention in the manufacture of a medicament for the treatment of SARS-CoV-2 or a disorder associated with SARS-CoV-2.

In a fifth aspect of the invention, there is provided a method of treating a disease or disorder susceptible to SARS-CoV-2 Mpro inhibition in a subject in need thereof, said method comprising administering to said subject a pharmaceutically effective amount of a compound of the invention, or salt, solvate, hydrate, N-oxide or prodrug thereof. A feature of this aspect is that the disease or disorder is SARS-CoV-2 or a disorder associated with SARS-CoV-2.

In view of the above, the compounds of the invention may be useful in preventing death or complications arising due to chronic underlying conditions or comorbidities in patients infected with SARS-CoV-2. Such chronic underlying conditions or comorbidities may include for example hypertension, obesity, chronic lung conditions (TB, asthma and cystic fibrosis), diabetes and cardiovascular conditions (coronary heart disease, congenital heart disease and heart failure).

In the above methods or uses, the compounds or compositions of the present invention may be administered in combination with one or more drugs which block cypP450 mediated metabolism. Said drugs may be selected from ritonavir, lopinavir or a combination thereof.

The term “treatment” and “treating” as used herein may include prophylaxis of the named disease or disorder, or amelioration or elimination of the disease or disorder once it has been established.

As used herein, the terms “administration” or “administering” mean a route of administration for a compound disclosed herein. Exemplary routes of administration include, but are not limited to, oral, intravenous, intraperitoneal, intraarterial, and intramuscular. The preferred route of administration can vary depending on various factors, e.g. the components of the pharmaceutical composition comprising a compound disclosed herein, site of the potential or actual disease and severity of disease.

The terms “subject” and “patient” are used herein interchangeably. They refer to a human or another mammal (e.g., mouse, rat, rabbit, dog, cat, cattle, swine, sheep, horse or primate) that can be afflicted with or is susceptible to a disease or condition but may or may not have the disease or condition. It is preferred that the subject is human.

“A therapeutically effective amount” refers to an amount of a compound of the invention that confers a therapeutic effect on the treated subject. The therapeutic effect may be objective (i.e. measurable by some test or marker) or subjective (i.e. subject gives an indication of or feels an effect).

Methods delineated herein include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

In other aspects, the methods herein include those further comprising monitoring subject response to the treatment administrations. Such monitoring may include periodic sampling of subject tissue, fluids, specimens, cells, proteins, chemical markers, genetic materials, etc. as markers or indicators of the treatment regimen. In other methods, the subject is pre-screened or identified as in need of such treatment by assessment for a relevant marker or indicator of suitability for such treatment.

Compounds of the invention may be for use as a single agent or in combination with one or more additional pharmaceutical agents. For examples, the compounds of the invention may be co-administered with HIV drugs which are known to block cypP450 mediated metabolism, such as ritonavir or a combination of lopinavir/ritonavir.

### Preparation of Compounds of the Invention

The compounds of the invention may be prepared by, or in analogy with, conventional methods. The preparation of intermediates and compounds according to the examples of the present invention may in particular be illuminated by the following Schemes. Definitions of variables in the structures in schemes herein are commensurate with those of corresponding positions in the formulae delineated herein.

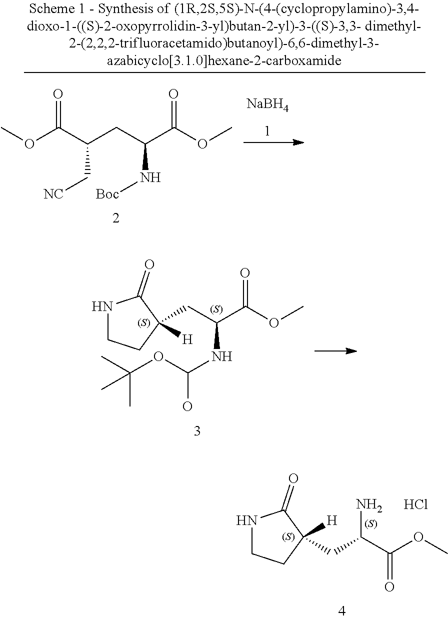

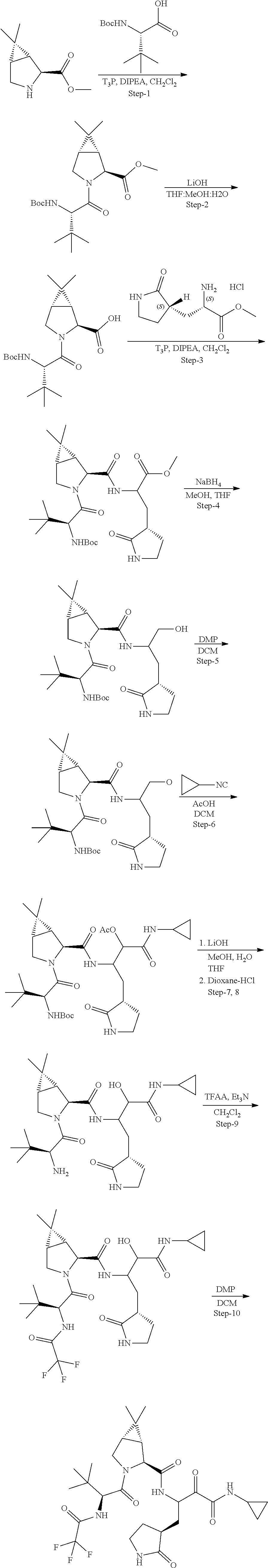

### Intermediate 1

### Synthesis of methyl (1R,2S,5S)-3-((S)-2-((tert-butoxycarbonyl)amino)-3,3-dimethylbutanoyl)-6,6-dimethyl-3-azabicyclo[3.1.0]hexane-2-carboxylate

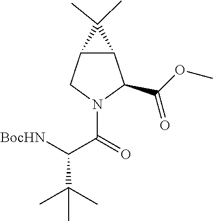 [SMILES: COC(=O)[C@@H]1[C@@H]2[C@H](CN1C(=O)[C@@H](NC(=O)OC(C)(C)C)C(C)(C)C)C2(C)C]

To a stirred solution of methyl (1S,2S,5R)-6,6-dimethyl-3-azabicyclo[3.1.0]hexane-2-carboxylate (1 g, 5.85 mmol) and (S)-2-((tert-butoxycarbonyl)amino)-3,3-dimethylbutanoic (1.36 g, 5.85 mmol) in DCM (20 mL) at 0° C., DIPEA (3.31 mL, 17.55 mmol) and propylphosphonic anhydride solution (≥50 wt. % in ethyl acetate, 5.22 mL, 8.78 mmol) were added and the resulting reaction mixture was stirred 16 h at RT. After completion of the reaction (monitored by LCMS), the reaction mixture was quenched with water (20 mL) and the aqueous layer was extracted with DCM (2×30 mL). The combined organic layer was washed with saturated NaHCO₃ (25 mL), brine solution (25 mL) and dried over anhydrous Na₂SO₄. The organic layer was concentrated under reduced pressure and the obtained crude was purified by Flash column chromatography (silica gel: 230-400 mesh, eluent: 50% EtOAc/pet-ether) to afford the methyl (1R,2S,5S)-3-((S)-2-((tert-butoxycarbonyl)amino)-3,3-dimethylbutanoyl)-6,6-dimethyl-3-azabicyclo[3.1.0]hexane-2-carboxylate as sticky colourless gum (1 g, 44.2%). LCMS (ELSD): 383.3 [MH]⁺.

### Intermediate 2

### Synthesis of (1R,2S,5S)-3-((S)-2-((tert-butoxycarbonyl)amino)-3,3-dimethylbutanoyl)-6,6-dimethyl-3-azabicyclo[3.1.0]hexane-2-carboxylic acid

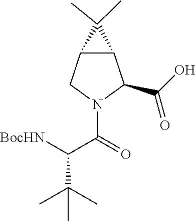 [SMILES: CC(C)(C)OC(=O)N[C@H](C(=O)N1C[C@H]2[C@@H]([C@H]1C(=O)O)C2(C)C)C(C)(C)C]

To a stirred solution of methyl (1R,2S,5S)-3-((S)-2-((tert-butoxycarbonyl)amino)-3,3-dimethylbutanoyl)-6,6-dimethyl-3-azabicyclo[3.1.0]hexane-2-carboxylate (1 g, 2.59 mmol) in a mixture of THF (4 mL), MeCOH (4 mL) and water (2 mL), LiOH·H2O (0.33 g, 7.76 mmol) was added at RT and the resulting reaction mixture was stirred 16 h at RT. After completion of the reaction (monitored by LCMS), the reaction mixture was concentrated under vacuum. The crude residue was diluted with water (5 mL), acidified with citric acid solution (2.5 mL) and the aqueous layer was extracted with DCM (2×10 mL). The Combined organic layer was washed with brine solution (25 mL), dried over anhydrous Na2SO4 and concentrated under reduced pressure to afford the (1R,2S,5S)-3-((S)-2-((tert-butoxycarbonyl)amino)-3,3-dimethylbutanoyl)-6,6-dimethyl-3-azabicyclo[3.1.0]hexane-2-carboxylic acid as yellow gum which was forwarded as such to the next step without any further purification (0.9 g, crude). LCMS (ELSD): 369.3 [MH]⁺. δ 12.53 (bs, 1H), 6.68 (d, J=9.6 Hz, 1H), 4.13 (s, 1H), 4.05 (d, J=9.6 Hz, 1H), 3.91 (d, J=10.4 Hz, 1H), 3.79-3.74 (m, 1H), 1.51-1.47 (m, 1H), 1.41-1.37 (m, 1H), 1.35 (s, 9H), 1.01 (s, 3H), 0.94 (s, 9H), 0.84 (s, 3H).

### Intermediate 3

### Synthesis of methyl 2-((1S,2S,5R)-3-((S)-2-((tert-butoxycarbonyl)amino)-3,3-dimethylbutanoyl)-6,6-dimethyl-3-azabicyclo[3.1.0]hexane-2-carboxamido)-3-((S)-2-oxopyrrolidin-3-yl)propanoate

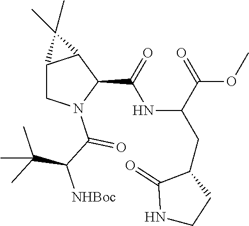

To a suspension of (1S,2S,5R)-3-((S)-2-((tert-butoxycarbonyl)amino)-3,3-dimethylbutanoyl)-6,6-dimethyl-3-azabicyclo[3.1.0]hexane-2-carboxylic acid (900 mg, 2.44 mmol), methyl (S)-2-amino-3-((S)-2-oxopyrrolidin-3-yl)propanoate hydrochloride (544 mg, 2.44 mmol) in DCM (20 mL) at 0° C., DIPEA (1.44 mL, 7.82 mmol) and propylphosphonic anhydride solution (≥50 wt. % in ethyl acetate, 1.45 mL, 2.44 mmol) were added slowly and the resulting reaction mixture was stirred 3 h at RT. After completion of the reaction (monitored by LCMS), the reaction mixture was quenched with saturated NaHCO₃ solution (15 mL) and diluted with water (20 mL). The aqueous layer was extracted with DCM (2×50 mL), then the combined organic layer was washed with brine solution (25 mL) and dried over anhydrous Na₂SO₄. The organic part was concentrated under reduced pressure and the resulting crude was purified by Flash column chromatography (silica gel: 230-400 mesh, eluent: 100% EtOAc) to afford the desired product methyl 2-((1S,2S,5R)-3-((S)-2-((tert-butoxycarbonyl)amino)-3,3-dimethylbutanoyl)-6,6-dimethyl-3-azabicyclo[3.1.0]hexane-2-carboxamido)-3-((S)-2-oxopyrrolidin-3-yl)propanoate as white solid (0.5 g, 35.9%). LCMS (ELSD): 537.3 [MH]⁺. δ 8.57 (d, J=8.4 Hz, 1H), 7.58 (s, 1H), 6.62 (d, J=9.0 Hz, 1H), 4.44-4.39 (m, 1H), 4.25 (s, 1H), 4.05-4.00 (m, 1H), 3.90-3.81 (m, 2H), 3.64 (s, 3H), 3.17-3.02 (m, 2H), 2.50-2.44 (m, 1H), 2.12-2.00 (m, 2H), 1.60-1.47 (m, 3H), 1.35 (s, 9H), 1.26-1.16 (m, 1H), 1.03 (s, 3H), 0.96-0.84 (m, 12H).

### Intermediate 4

### Synthesis of tert-butyl ((2S)-1-((1S,2S,5R)-2-((1-hydroxy-3-((S)-2-oxopyrrolidin-3-yl)propan-2-yl)carbamoyl)-6,6-dimethyl-3-azabicyclo[3.1.0]hexan-3-yl)-3,3-dimethyl-1-oxobutan-2-yl)carbamate

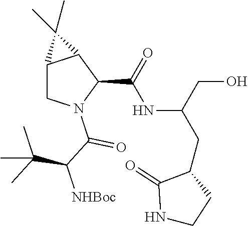

To a suspension of methyl 2-((1S,2S,5R)-3-((S)-2-((tert-butoxycarbonyl)amino)-3,3-dimethylbutanoyl)-6,6-dimethyl-3-azabicyclo[3.1.0]hexane-2-carboxamido)-3-((S)-2-oxopyrrolidin-3-yl)propanoate (500 mg, 0.93 mmol) in MeOH (5 mL) and THF (10 mL) at 0° C., NaBH₄ (211 mg, 5.59 mmol) was slowly added portion wise and the resulting reaction mixture was stirred 3 h at 0° C. After completion of the reaction (monitored by LCMS), the reaction mixture was concentrated under reduced pressure. The obtained residue was partitioned between EtOAc (10 mL) and water (10 mL). The aqueous layer was extracted with EtOAc (2×20 mL) and the combined organic layer was washed with brine solution (25 mL), dried over anhydrous Na₂SO₄ and concentrated under reduced pressure. The resulting crude was purified by flash column chromatography (silica gel: 230-400 mesh; eluent: 0-10% MeCOH/DCM) to afford the tert-butyl ((2S)-1-((1S,2S,5R)-2-((1-hydroxy-3-((S)-2-oxopyrrolidin-3-yl)propan-2-yl)carbamoyl)-6,6-dimethyl-3-azabicyclo[3.1.0]hexan-3-yl)-3,3-dimethyl-1-oxobutan-2-yl)carbamate as a white solid (0.3 g, 60.8%). LCMS (ELSD): 509.4 [MH]⁺. δ 7.80 (d, J=9.6 Hz, 1H), 7.45 (s, 1H), 6.61 (d, J=9.2 Hz, 1H), 4.66 (t, J=5.6 Hz, 1H), 4.18 (s, 1H), 4.04-3.99 (m, 1H), 3.90-3.75 (m, 3H), 3.30-3.27 (m, 2H), 3.12-3.08 (m, 1H), 2.99-2.92 (m, 1H), 2.52-2.38 (m, 1H), 2.20-2.10 (m, 1H), 1.73-1.68 (m, 1H), 1.52-1.35 (m, 13H), 0.98-0.71 (m, 15H).

### Intermediate 5

### Synthesis of tert-butyl ((2S)-1-((1S,2S,5R)-6,6-dimethyl-2-((1-oxo-3-((S)-2-oxopyrrolidin-3-yl)propan-2-yl)carbamoyl)-3-azabicyclo[3.1.0]hexan-3-yl)-3,3-dimethyl-1-oxobutan-2-yl)carbamate

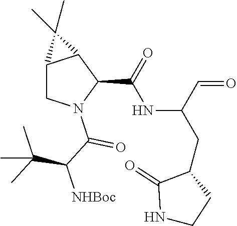

To a suspension of tert-butyl ((2S)-1-((1S,2S,5R)-2-((1-hydroxy-3-((S)-2-oxopyrrolidin-3-yl)propan-2-yl)carbamoyl)-6,6-dimethyl-3-azabicyclo[3.1.0]hexan-3-yl)-3,3-dimethyl-1-oxobutan-2-yl)carbamate (300 mg, 0.59 mmol) in DCM (10 mL) at 0° C., DMP (500 mg, 1.18 mmol) was added slowly and the resulting reaction mixture was stirred 3 h at RT. After completion of reaction (monitored by UPLCMS & LCMS), the reaction mixture was quenched with saturated NaHCO₃ (10 mL) and diluted with water (15 mL). The aqueous layer was extracted with DCM (2×25 mL) and the combined organic layer was washed with saturated NaHCO₃ (20 mL), brine solution (20 mL) and dried over anhydrous Na₂SO₄. The organic part was concentrated under reduced pressure and the resulting crude was purified by Flash column chromatography (silica gel: 230-400 mesh, eluent: 10% MeCOH/DCM) to afford the desired compound tert-butyl ((2S)-1-((1S,2S,5R)-6,6-dimethyl-2-((1-oxo-3-((S)-2-oxopyrrolidin-3-yl)propan-2-yl)carbamoyl)-3-azabicyclo[3.1.0]hexan-3-yl)-3,3-dimethyl-1-oxobutan-2-yl)carbamate as an off-white solid (0.3 g, 82%). LCMS: 507.4 [MH]⁺.

### Intermediate 6

### Synthesis of 3-((1S,2S,5R)-3-((S)-2-((tert-butoxycarbonyl)amino)-3,3-dimethylbutanoyl)-6,6-dimethyl-3-azabicyclo[3.1.0]hexane-2-carboxamido)-1-(cyclopropylamino)-1-oxo-4-((S)-2-oxopyrrolidin-3-yl)butan-2-yl acetate

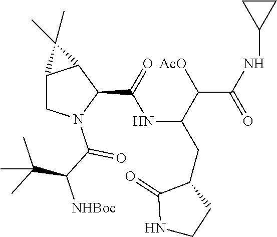

To a suspension of tert-butyl ((2S)-1-((1S,2S,5R)-6,6-dimethyl-2-((1-oxo-3-((S)-2-oxopyrrolidin-3-yl)propan-2-yl)carbamoyl)-3-azabicyclo[3.1.0]hexan-3-yl)-3,3-dimethyl-1-oxobutan-2-yl)carbamate (300 mg, 0.59 mmol) in DCM (3 mL) at 0° C., acetic acid (0.07 mL, 1.18 mmol), isocyanocyclopropane (0.039 mL, 0.59 mmol) were slowly added and the resulting reaction mixture was stirred 16 h at RT. After completion of the reaction (monitored by UPLCMS & LCMS), the reaction mixture was quenched with water (10 mL) and the aqueous layer was extracted with DCM (2×20 mL). Then the combined organic layer was washed with brine solution (20 mL), dried over anhydrous Na₂SO₄, concentrated under reduced pressure. The resulting crude Purified by Flash column chromatography (Silica gel: 230-400 mesh, eluent: 10% MeCOH/DCM) to afford the 3-((1S,2S,5R)-3-((S)-2-((tert-butoxycarbonyl)amino)-3,3-dimethylbutanoyl)-6,6-dimethyl-3-azabicyclo[3.1.0]hexane-2-carboxamido)-1-(cyclopropylamino)-1-oxo-4-((S)-2-oxopyrrolidin-3-yl)butan-2-yl acetate as yellow gum (0.3 g, 70.3%). LCMS: 634.4 [MH]⁺.

### Intermediate 7

### Synthesis of tert-butyl ((2S)-1-((1S,2S,5R)-2-((4-(cyclopropylamino)-3-hydroxy-4-oxo-1-((S)-2-oxopyrrolidin-3-yl)butan-2-yl)carbamoyl)-6,6-dimethyl-3-azabicyclo[3.1.0]hexan-3-yl)-3,3-dimethyl-1-oxobutan-2-yl)carbamate

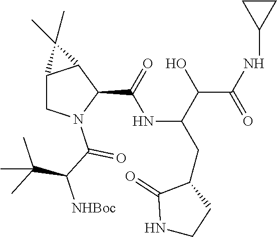

To a suspension of 3-((1S,2S,5R)-3-((S)-2-((tert-butoxycarbonyl)amino)-3,3-dimethylbutanoyl)-6,6-dimethyl-3-azabicyclo[3.1.0]hexane-2-carboxamido)-1-(cyclopropylamino)-1-oxo-4-((S)-2-oxopyrrolidin-3-yl)butan-2-yl acetate (300 mg, 0.379 mmol) in a mixture of THF (2 mL), MeCOH (2 mL) and water (1 mL) at 0° C., LiOH·H₂O (48.2 mg, 1.13 mmol) was added and the resulting reaction mixture was stirred 3 h at 0° C. After completion of the reaction (monitored by UPLCMS & LCMS), the reaction mixture was diluted with water (10 mL) and the aqueous layer was extracted with DCM (2×15 mL). The combined organic layer was washed with brine solution (20 mL), dried over anhydrous Na₂SO₄ and concentrated under reduced pressure. The resulting crude was purified by Flash Column chromatography (silica gel: 230-400 mesh, eluent: 10% MeCOH/DCM) to afford the desired compound tert-butyl ((2S)-1-((1S,2S,5R)-2-((4-(cyclopropylamino)-3-hydroxy-4-oxo-1-((S)-2-oxopyrrolidin-3-yl)butan-2-yl)carbamoyl)-6,6-dimethyl-3-azabicyclo[3.1.0]hexan-3-yl)-3,3-dimethyl-1-oxobutan-2-yl)carbamate as brown solid (0.15 g, 64.3%). LCMS (ELSD): 592.4 [MH]⁺.

### Example 1

### Synthesis of (1R,2S,5S)—N-(4-(cyclopropylamino)-3,4-dioxo-1-((S)-2-oxopyrrolidin-3-yl)butan-2-yl)-3-((S)-3,3-dimethyl-2-(2,2,2-trifluoroacetamido)butanoyl)-6,6-dimethyl-3-azabicyclo[3.1.0]hexane-2-carboxamide

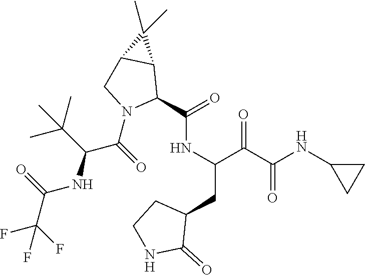 [SMILES: CC(C)(C)[C@H](NC(=O)C(F)(F)F)C(=O)N1C[C@H]2[C@@H]([C@H]1C(=O)NC(C[C@@H]1CCNC1=O)C(=O)C(=O)NC1CC1)C2(C)C]

To a suspension of (1S,2S,5R)—N-(4-(cyclopropylamino)-3-hydroxy-4-oxo-1-((S)-2-oxopyrrolidin-3-yl)butan-2-yl)-3-((S)-3,3-dimethyl-2-(2,2,2-trifluoroacetamido) butanoyl)-6,6-dimethyl-3-azabicyclo[3.1.0]hexane-2-carboxamide (100 mg, 0.17 mmol) in DCM (5 mL) at 0° C., DMP (144 mg, 0.34 mmol) was added portionwise and the resulting reaction mixture was stirred 3 h at RT. After completion of the reaction (monitored by LCMS), the reaction mixture was diluted with water (10 mL) and the aqueous layer was extracted with DCM (2×20 mL). The combined organic layer was washed with saturated NaHCO₃ (20 mL), brine solution (20 mL), dried over anhydrous Na₂SO₄ and concentrated under reduced pressure. The crude was purified by Prep-HPLC (Method A). The fractions were collected and concentrated under reduced pressure. The obtained residue was partitioned between 10% NaHCO₃ solution (20 mL) and DCM (2×20 mL). The organic layer was separated, dried over anhydrous Na₂SO₄, concentrated under reduced pressure and lyophilized to obtain the desired compound (1R,2S,5S)—N-(4-(cyclopropylamino)-3,4-dioxo-1-((S)-2-oxopyrrolidin-3-yl)butan-2-yl)-3-((S)-3,3-dimethyl-2-(2,2,2-trifluoroacetamido)butanoyl)-6,6-dimethyl-3-azabicyclo[3.1.0]hexane-2-carboxamide as a pale yellow solid (0.051 g, 50.8%). LCMS (ELSD): 586.3 [MH]⁺. δ 9.40-5.94 (m, 4H), 4.42 (d, J=8.4 Hz, 1H), 4.29-4.25 (m, 1H), 4.10-3.84 (m, 1H), 3.69-3.53 (m, 1H), 3.20-2.95 (m, 2H), 2.80-2.52 (m, 2H), 2.31-2.14 (m, 1H), 1.90-1.36 (m, 6H), 1.03-0.92 (m, 12H), 0.90-0.68 (m, 3H), 0.68-0.41 (m, 4H).

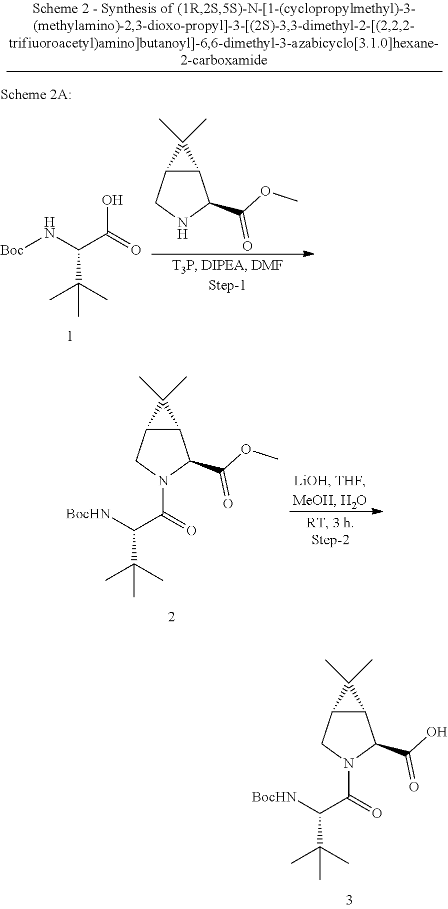

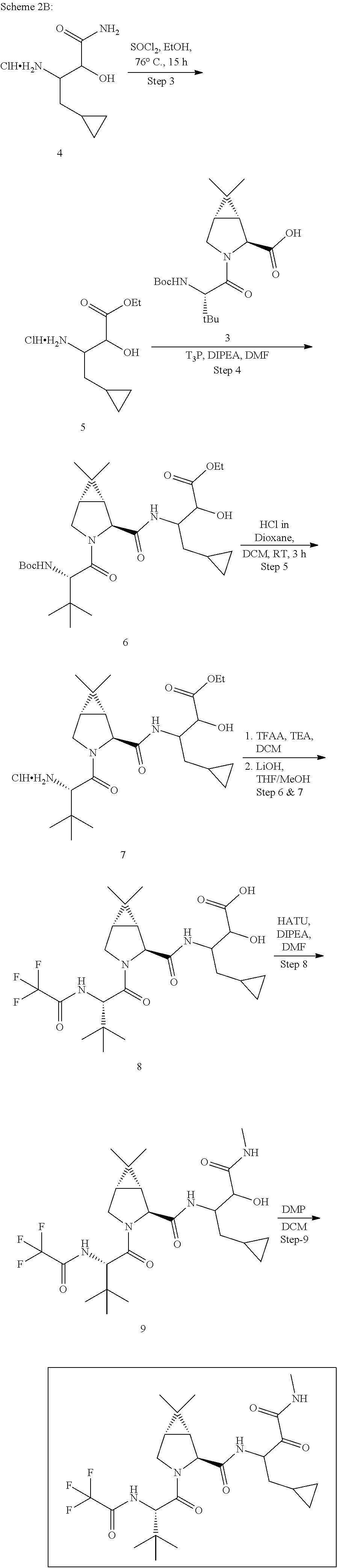

### Intermediate 8

### Synthesis of methyl (1R,2S,5S)-3-((S)-2-((tert-butoxycarbonyl)amino)-3,3-dimethylbutanoyl)-6,6-dimethyl-3-azabicyclo[3.1.0]hexane-2-carboxylate

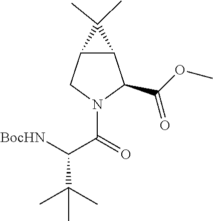 [SMILES: COC(=O)[C@@H]1[C@@H]2[C@H](CN1C(=O)[C@@H](NC(=O)OC(C)(C)C)C(C)(C)C)C2(C)C]

To a suspension of methyl (1R,2S,5S)-6,6-dimethyl-3-azabicyclo[3.1.0]hexane-2-carboxylate (2 g, 11.70 mmol) and (S)-2-((tert-butoxycarbonyl)amino)-3,3-dimethylbutanoic acid (2.73 g, 11.70 mmol) in DCM (30 mL) was added DIPEA (2.1 mL, 11.70 mmol) at RT followed by T3P (6.83 mL, 11.70 mmol, 50% solution in EtOAc) at 0° C. The resultant reaction mixture was stirred 3 h at RT. Upon completion of the reaction (monitored by LCMS), the reaction was quenched with aq. 10% NaHCO₃ solution at and the aqueous layer was extracted with DCM (2×25 mL). The combined organic layer was dried over anhydrous Na₂SO₄ and concentrated under reduced pressure. The crude residue was purified by flash column chromatography using (silica gel: 230-400 mesh, eluent: 0-50% EtOAc/pet ether) to afford methyl (1R,2S,5S)-3-((S)-2-((tert-butoxycarbonyl)amino)-3,3-dimethylbutanoyl)-6,6-dimethyl-3-azabicyclo[3.1.0]hexane-2-carboxylate as a pale yellow gum (2.42 g, 52.7%). LCMS (ELSD): 383.4 [MH]⁺. δ 6.76 (d, J=9.6 Hz, 1H), 4.21 (s, 1H), 4.05 (d, J=9.2 Hz, 1H), 3.94 (d, J=10.4 Hz, 1H), 3.81-3.77 (m, 1H), 3.65 (s, 3H), 1.54-1.51 (m, 1H), 1.41-1.38 (m, 1H), 1.37 (s, 9H), 1.01 (s, 3H), 0.96 (s, 9H), 0.85 (s, 3H).

### Intermediate 9

### Synthesis of (1R,2S,5S)-3-((S)-2-((tert-butoxycarbonyl)amino)-3,3-dimethylbutanoyl)-6,6-dimethyl-3-azabicyclo[3.1.0]hexane-2-carboxylic acid

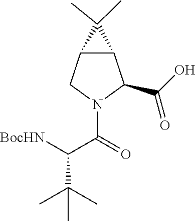 [SMILES: CC(C)(C)OC(=O)N[C@H](C(=O)N1C[C@H]2[C@@H]([C@H]1C(=O)O)C2(C)C)C(C)(C)C]

To a suspension of methyl (1R,2S,5S)-3-((S)-2-((tert-butoxycarbonyl)amino)-3,3-dimethylbutanoyl)-6,6-dimethyl-3-azabicyclo[3.1.0]hexane-2-carboxylate (2.5 g, 6.37 mmol) in a mixture of THF (20 mL), MeCOH (6 mL) and water (6 mL) was added LiOH·H₂O (0.15 g, 6.37 mmol) at RT. The resultant reaction mixture was stirred 3 h at RT. After completion of the reaction (monitored by LCMS), the reaction mixture was diluted with water (10 mL) and washed with MTBE (10 mL). The aqueous layer was acidified with HCl (1.5 N, pH ˜2-3) and extracted with DCM (2×30 mL). The combined organic layer was dried over anhydrous Na₂SO₄ and concentrated under reduced pressure to give (1R,2S,5S)-3-((S)-2-((tert-butoxycarbonyl)amino)-3,3-dimethylbutanoyl)-6,6-dimethyl-3-azabicyclo[3.1.0]hexane-2-carboxylic acid as a white solid which was used forwarded to the next step without any further purification (2.12 g, crude, 88%). LCMS (ELSD): 369.3 [MH]⁺. δ 12.53 (br s, 1H), 6.68 (d, J=9.6 Hz, 1H), 4.13 (s, 1H), 4.05 (d, J=9.6 Hz, 1H), 3.91 (d, J=10.4 Hz, 1H), 3.79-3.74 (m, 1H), 1.51-1.47 (m, 1H), 1.41-1.37 (m, 1H), 1.35 (s, 9H), 1.01 (s, 3H), 0.94 (s, 9H), 0.84 (s, 3H).

### Intermediate 10

### Synthesis of ethyl 3-amino-4-cyclopropyl-2-hydroxybutanoate

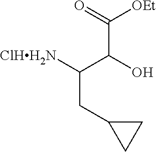

To a stirred solution of 3-amino-4-cyclopropyl-2-hydroxybutanamide (2.5 g, 15.64 mmol, HEP-WUXI-A81-INT5) in EtOH (20 mL) at 0° C., SOCl₂ (1.15 mL, 15.64 mmol) was added dropwise and the resulting reaction mixture was stirred 15 h at 65° C. After completion of the reaction (monitored by LCMS), the reaction mixture was concentrated under reduced pressure to remove volatiles. The residue was dried using azeotropic co-distillation with toluene (2×20 mL) to afford ethyl 3-amino-4-cyclopropyl-2-hydroxybutanoate as a brown semi-solid which was used in the following step without any further purification (2.1 g, crude, 63.1%). LCMS (ELSD): 188.3 [MH]⁺.

### Intermediate 11

### Synthesis of ethyl 3-((1R,2S,5S)-3-((S)-2-((tert-butoxycarbonyl)amino)-3,3-dimethylbutanoyl)-6,6-dimethyl-3-azabicyclo[3.1.0]hexane-2-carboxamido)-4-cyclopropyl-2-hydroxybutanoate

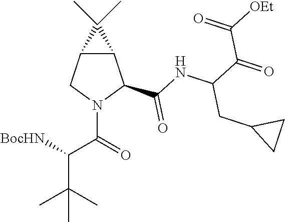 [SMILES: CCOC(=O)C(=O)C(CC1CC1)NC(=O)[C@@H]1[C@@H]2[C@H](CN1C(=O)[C@@H](NC(=O)OC(C)(C)C)C(C)(C)C)C2(C)C]

To a suspension of (1R,2S,5S)-3-((S)-2-((tert-butoxycarbonyl)amino)-3,3-dimethylbutanoyl)-6,6-dimethyl-3-azabicyclo[3.1.0]hexane-2-carboxylic acid (2.45 g, 6.25 mmol) and ethyl 3-amino-4-cyclopropyl-2-hydroxybutanoate (1.65 g, 7.50 mmol) in DCM (20 mL), DIPEA (3.37 mL, 18.75 mmol) and followed by T₃P (50% in EtOAc) (5.58 mL, 9.38 mmol) were added at 0° C. The resultant reaction mixture was stirred 3 h at RT. After completion of the reaction (monitored by LCMS), the reaction mixture was quenched with aq. NaHCO₃ (10%) at RT and the aqueous layer was extracted with DCM (2×25 mL). The combined organic layer was dried over anhydrous Na₂SO₄ and concentrated under reduced pressure. The crude residue was purified by flash column chromatography using (silica ge: 230-400 mesh, eluent: 0-50% EtOAc/pet-ether) to afford ethyl 3-((1R,2S,5S)-3-((S)-2-((tert-butoxycarbonyl)amino)-3,3-dimethylbutanoyl)-6,6-dimethyl-3-azabicyclo[3.1.0]hexane-2-carboxamido)-4-cyclopropyl-2-hydroxybutanoate as a pale yellow gum (2.3 g, 67%). LCMS (ELSD): 538.4 [MH]⁺. δ 7.90 (d, J=8.8 Hz, 1H), 6.57 (d, J=9.2 Hz, 1H), 5.57-5.50 (m, 1H), 4.11-3.71 (m, 8H), 1.51-1.41 (m, 2H), 1.40 (s, 9H), 1.05-0.80 (m, 18H), 0.72-0.61 (m, 1H), 0.35-0.03 (m, 5H), 0.14-0.05 (m, 1H).

### Intermediate 12

### Synthesis of ethyl 3-((1R,2S,5S)-3-((S)-2-amino-3,3-dimethylbutanoyl)-6,6-dimethyl-3-azabicyclo[3.1.0]hexane-2-carboxamido)-4-cyclopropyl-2-hydroxybutanoate hydrochloride

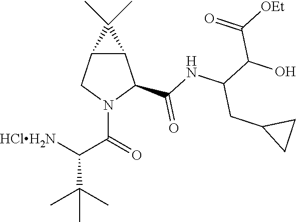

To a stirred solution of ethyl 3-((1R,2S,5S)-3-((S)-2-((tert-butoxycarbonyl)amino)-3,3-dimethylbutanoyl)-6,6-dimethyl-3-azabicyclo[3.1.0]hexane-2-carboxamido)-4-cyclopropyl-2-hydroxybutanoate (2.2 g, 4.01 mmol) in DCM (10 mL) at 0° C., HCl in dioxane (5 mL, 4 M) was added dropwise and the resulting reaction mixture was stirred 3 h at RT. After completion of the reaction (monitored by LCMS), the supernatant layer was decanted from the reaction mixture, and the solid gummy residue was triturated with MTBE (10 mL). The ether layer was decanted, and the residue was dried under vacuum to get ethyl 3-((1R,2S,5S)-3-((S)-2-amino-3,3-dimethylbutanoyl)-6,6-dimethyl-3-azabicyclo[3.1.0]hexane-2-carboxamido)-4-cyclopropyl-2-hydroxybutanoate hydrochloride as an off white solid, which was used in the next step without any further purification (1.8 g, 76%). LCMS (ELSD): 438.4 [MH]⁺.

### Intermediate 13

### Synthesis of ethyl 4-cyclopropyl-3-((1R,2S,5S)-3-((S)-3,3-dimethyl-2-(2,2,2-trifluoroacetamido)butanoyl)-6,6-dimethyl-3-azabicyclo[3.1.0]hexane-2-carboxamido)-2-hydroxybutanoate

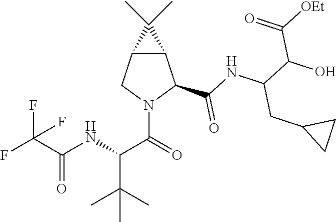 [SMILES: CCOC(=O)C(O)C(CC1CC1)NC(=O)[C@@H]1[C@@H]2[C@H](CN1C(=O)[C@@H](NC(=O)C(F)(F)F)C(C)(C)C)C2(C)C]

To a suspension of ethyl 3-((1R,2S,5S)-3-((S)-2-amino-3,3-dimethylbutanoyl)-6,6-dimethyl-3-azabicyclo[3.1.0]hexane-2-carboxamido)-4-cyclopropyl-2-hydroxybutanoate hydrochloride (0.47 g, 0.99 mmol) in DCM (10 mL) at 0° C., triethyl amine (0.34 mL, 2.47 mmol) and trifluoroacetic anhydride (0.17 mL, 1.19 mmol) were added and the resultant reaction mixture was stirred 2 h at RT. After completion of the reaction (monitored by LCMS), the reaction mixture was diluted with water (15 mL) and the aqueous layer was extracted with DCM (3×10 mL). The combined organic layer was washed with saturated aqueous NaHCO₃ solution (5 mL) followed by brine (10 mL) and dried over anhydrous Na₂SO₄. The organic part was filtered and concentrated under reduced pressure to afford the desired compound ethyl 4-cyclopropyl-3-((1R,2S,5S)-3-((S)-3,3-dimethyl-2-(2,2,2-trifluoroacetamido)butanoyl)-6,6-dimethyl-3-azabicyclo[3.1.0]hexane-2-carboxamido)-2-hydroxybutanoate. This was forwarded to the next step without any further purification (0.53 g, 97%). LCMS (ELSD): 534.3 [MH]⁺.

### Intermediate 14

### Synthesis of 4-cyclopropyl-3-((1R,2S,5S)-3-((S)-3,3-dimethyl-2-(2,2,2-trifluoroacetamido)butanoyl)-6,6-dimethyl-3-azabicyclo[3.1.0]hexane-2-carboxamido)-2-hydroxybutanoic acid

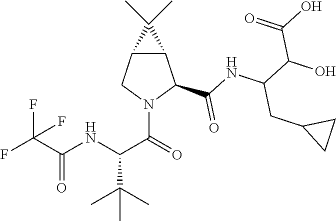 [SMILES: CC(C)(C)[C@H](NC(=O)C(F)(F)F)C(=O)N1C[C@H]2[C@@H]([C@H]1C(=O)NC(CC1CC1)C(O)C(=O)O)C2(C)C]

To a suspension of ethyl 4-cyclopropyl-3-((1R,2S,5S)-3-((S)-3,3-dimethyl-2-(2,2,2-trifluoroacetamido)butanoyl)-6,6-dimethyl-3-azabicyclo[3.1.0]hexane-2-carboxamido)-2-hydroxybutanoate (530 mg, 0.99 mmol) in a mixture of THF (5 mL), MeCOH (4 mL) and water (3 mL), LiOH (71.4 mg, 2.98 mmol) was added at RT and the resulting reaction mixture was stirred 2 h at RT. After completion of the reaction (monitored by LCMS), the reaction mixture was diluted with ice cold water (20 mL) and the aqueous layer was washed with EtOAc (2×15 mL). The combined aqueous extract was acidified with 1.5 N HCl solution (10 mL). The obtained solid was filtered through buchner funnel and concentrated under reduced pressure to afford 4-cyclopropyl-3-((1R,2S,5S)-3-((S)-3,3-dimethyl-2-(2,2,2-trifluoroacetamido)butanoyl)-6,6-dimethyl-3-azabicyclo[3.1.0]hexane-2-carboxamido)-2-hydroxybutanoic acid which was forwarded to the next step as such without any further purification (0.36 g, 71%). LCMS: 506.4 [MH]⁺.

### Intermediate 15

### Synthesis of (1R,2S,5S)—N-(1-cyclopropyl-3-hydroxy-4-(methylamino)-4-oxobutan-2-yl)-3-((S)-3,3-dimethyl-2-(2,2,2-trifluoroacetamido)butanoyl)-6,6-dimethyl-3-azabicyclo[3.1.0]hexane-2-carboxamide

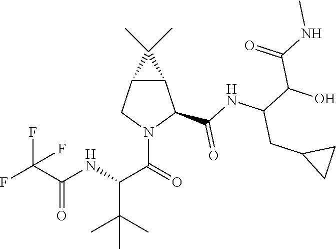 [SMILES: CNC(=O)C(O)C(CC1CC1)NC(=O)[C@@H]1[C@@H]2[C@H](CN1C(=O)[C@@H](NC(=O)C(F)(F)F)C(C)(C)C)C2(C)C]

To a suspension of 4-cyclopropyl-3-((1R,2S,5S)-3-((S)-3,3-dimethyl-2-(2,2,2-trifluoroacetamido)butanoyl)-6,6-dimethyl-3-azabicyclo[3.1.0]hexane-2-carboxamido)-2-hydroxybutanoic acid (360 mg, 0.71 mmol) in DMF (5 mL) at 0° C., DIPEA (0.32 mL, 1.78 mmol) and HATU (406 mg, 1.07 mmol) were added. Then MeNH₂ in THF (0.53 mL, 1.07 mmol) was added and the reaction mixture was stirred 2 h at RT. After completion of the reaction (monitored by UPLC-MS), the reaction mixture was diluted with water (15 mL) and the aqueous layer was extracted with DCM (3×10 mL). The combined organic layer was washed with brine (10 mL), dried over anhydrous Na₂SO₄, filtered and concentrated under reduced pressure. The crude residue was purified by flash column chromatography (silica-gel: 230-400 mesh, eluent: 20-80% of EtOAc/per-ether) to obtain the desired product (1R,2S,5S)—N-(1-cyclopropyl-3-hydroxy-4-(methylamino)-4-oxobutan-2-yl)-3-((S)-3,3-dimethyl-2-(2,2,2-trifluoroacetamido)butanoyl)-6,6-dimethyl-3-azabicyclo[3.1.0]hexane-2-carboxamide as a yellowish liquid (0.31 g, 76%). LCMS (ELSD): 519.3 [MH]⁺.

### Example 2

### Synthesis of (1R,2S,5S)—N-[1-(cyclopropylmethyl)-3-(methylamino)-2,3-dioxo-propyl]-3-[(2S)-3,3-dimethyl-2-[(2,2,2-trifluoroacetyl)amino]butanoyl]-6,6-dimethyl-3-azabicyclo[3.1.0]hexane-2-carboxamide

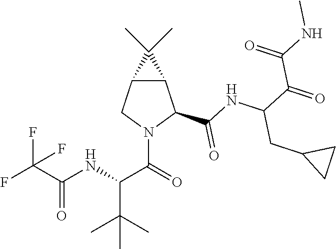 [SMILES: CNC(=O)C(=O)C(CC1CC1)NC(=O)[C@@H]1[C@@H]2[C@H](CN1C(=O)[C@@H](NC(=O)C(F)(F)F)C(C)(C)C)C2(C)C]

To a suspension of (1R,2S,5S)—N-(1-cyclopropyl-3-hydroxy-4-(methylamino)-4-oxobutan-2-yl)-3-((S)-3,3-dimethyl-2-(2,2,2-trifluoroacetamido)butanoyl)-6,6-dimethyl-3-azabicyclo[3.1.0]hexane-2-carboxamide (0.31 g, 0.59 mmol) in DCM (8 mL), DMP (0.51 g, 1.19 mmol) was added portion wise at Rt and the resulting reaction mixture was stirred 1.5 h at RT. After completion of the reaction (monitored by LCMS), the reaction mixture was diluted with ice cold water (15 mL) and the aqueous layer was extracted with DCM (3×10 mL) The combined organic layer was washed with saturated NaHCO₃ solution (10 mL), brine solution (10 mL) and dried over anhydrous Na₂SO₄. The organic part was concentrated under reduced pressure and the resulting crude was purified by Prep-HPLC (method A: Method: A-0.1% TFA in water, B-ACN, Flow rate: 2.0 mL/min, column: X-Bridge C8). The fractions were concentrated and washed with saturated aqueous NaHCO₃ solution (10 mL). The aqueous layer was extracted with DCM (3×15 mL), dried over anhydrous Na₂SO₄ and concentrated under reduced pressure. The obtained compound was dried and lyophilised to afford the desired product (1R,2S,5S)—N-[1-(cyclopropylmethyl)-3-(methylamino)-2,3-dioxo-propyl]-3-[(2S)-3,3-dimethyl-2-[(2,2,2-trifluoroacetyl)amino]butanoyl]-6,6-dimethyl-3-azabicyclo[3.1.0]hexane-2-carboxamide as an off-white solid (0.056 g, 24.67%). LCMS (ELSD): 517.3 [MH]⁺. δ 9.47-9.30 (m, 1H), 8.65-8.47 (m, 1H), 6.22-5.90 (m, 1H), 5.10-4.90 (m, 1H), 4.43-4.29 (m, 2H), 3.90-3.70 (m, 1H), 3.73-3.67 (m, 1H), 2.78-2.50 (m, 3H), 1.70-1.40 (m, 2H), 0.90-0.63 (m, 16H), 0.45-−0.14 (m, 6H).

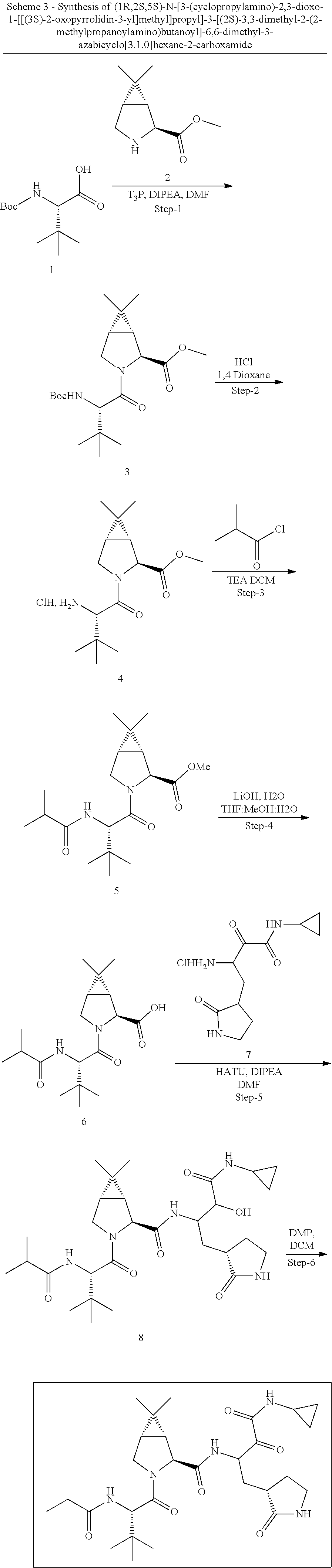

### Intermediate 16

### Synthesis of methyl (1R,2S,5S)-3-((S)-2-((tert-butoxycarbonyl)amino)-3,3-dimethylbutanoyl)-6,6-dimethyl-3-azabicyclo[3.1.0]hexane-2-carboxylate

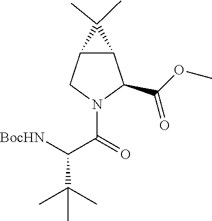 [SMILES: COC(=O)[C@@H]1[C@@H]2[C@H](CN1C(=O)[C@@H](NC(=O)OC(C)(C)C)C(C)(C)C)C2(C)C]

To a suspension of (S)-2-((tert-butoxycarbonyl)amino)-3,3-dimethylbutanoic acid (1.8 g, 7.78 mmol)) and methyl (1R,2S,5S)-6,6-dimethyl-3-azabicyclo[3.1.0]hexane-2-carboxylate (1.317 g, 7.78 mmol) in DCM (20 mL), DIPEA (4.08 mL, 23.35 mmol) was added at 0° C. Then Propylphosphonic anhydride solution≥50 wt. % in ethyl acetate (7.01 mL, 11.67 mmol) was added and the resulting reaction mixture was stirred at RT for 16 h. After completion of the reaction (monitored by UPLC-LCMS), the reaction mass was diluted with 10% NaHCO₃ solution (20 mL) and the aqueous layer was extracted with DCM (3×20 mL). The combined organic layer was separated, dried over anhydrous Na₂SO₄ and concentrated under reduced pressure. The resulting crude compound was purified by Biotage-Isolera using 25 g silica snap (silica gel: 230-400 mesh, eluent: 0-30% Ethyl acetate in pet-ether as gradient) to afford the desired compound methyl (1R,2S,5S)-3-((S)-2-((tert-butoxycarbonyl)amino)-3,3-dimethylbutanoyl)-6,6-dimethyl-3-azabicyclo[3.1.0]hexane-2-carboxylate as a yellow gum (1.8 g, 58.0%). LCMS (ELSD): 383.4 [MH]⁺.

### Intermediate 17

### Synthesis of methyl (1R,2S,5S)-3-((S)-2-amino-3,3-dimethylbutanoyl)-6,6-dimethyl-3-azabicyclo[3.1.0]hexane-2-carboxylate hydrochloride

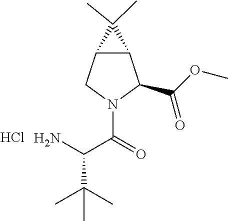 [SMILES: Cl ; COC(=O)[C@@H]1[C@@H]2[C@H](CN1C(=O)[C@@H](N)C(C)(C)C)C2(C)C]

To a suspension of methyl (1R,2S,5S)-3-((S)-2-((tert-butoxycarbonyl)amino)-3,3-dimethylbutanoyl)-6,6-dimethyl-3-azabicyclo[3.1.0]hexane-2-carboxylate (500 mg, 1.30 mmol) in 1,4-Dioxane (5 mL), HCl-dioxane (4 M, 5 mL, 20.00 mmol) was added dropwise at 0° C. and the reaction mixture was stirred 5 h at RT. After completion of the reaction (monitored by UPLC-LCMS), the reaction mixture was concentrated under reduced pressure to afford the desired compound methyl (1R,2S,5S)-3-((S)-2-amino-3,3-dimethylbutanoyl)-6,6-dimethyl-3-azabicyclo[3.1.0]hexane-2-carboxylate hydrochloride as brown gum. This compound was forwarded as such to the next step without any further purification (0.500 g, crude). LCMS (ELSD): 283.4 [MH]⁺.

### Intermediate 18

### Synthesis of methyl (1R,2S,5S)-3-((S)-2-isobutyramido-3,3-dimethylbutanoyl)-6,6-dimethyl-3-azabicyclo[3.1.0]hexane-2-carboxylate

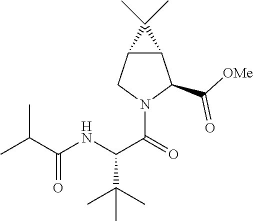 [SMILES: COC(=O)[C@@H]1[C@@H]2[C@H](CN1C(=O)[C@@H](NC(=O)C(C)C)C(C)(C)C)C2(C)C]

To a suspension of methyl (1R,2S,5S)-3-((S)-2-amino-3,3-dimethylbutanoyl)-6,6-dimethyl-3-azabicyclo[3.1.0]hexane-2-carboxylate hydrochloride (500 mg, 1.56 mmol) in DCM (5 mL) at 0° C., triethylamine (0.6 mL, 4.27 mmol) and isobutyryl chloride (0.2 mL, 1.91 mmol) were added and the reaction mixture was stirred 4 h at RT. After completion of the reaction (monitored by LCMS), the reaction mixture was concentrated under reduced pressure to afford the desired compound methyl (1R,2S,5S)-3-((S)-2-isobutyramido-3,3-dimethylbutanoyl)-6,6-dimethyl-3-azabicyclo[3.1.0]hexane-2-carboxylate as yellow gum. This compound was forwarded to the next step as such without any further purification (0.478 g, crude). LCMS (ELSD): 353.3 [MH]⁺.

### Intermediate 19

### Synthesis of (1R,2S,5S)-3-((S)-2-isobutyramido-3,3-dimethylbutanoyl)-6,6-dimethyl-3-azabicyclo[3.1.0]hexane-2-carboxylic acid

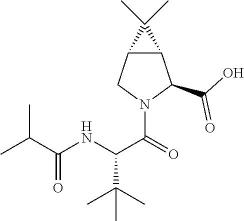 [SMILES: CC(C)C(=O)N[C@H](C(=O)N1C[C@H]2[C@@H]([C@H]1C(=O)O)C2(C)C)C(C)(C)C]

To a suspension of methyl (1R,2S,5S)-3-((S)-2-isobutyramido-3,3-dimethylbutanoyl)-6,6-dimethyl-3-azabicyclo[3.1.0]hexane-2-carboxylate (470 mg, 1.33 mmol) in a mixture of THF (3 mL), water (2 mL) and methanol (1 mL), LiOH·H₂O (168 mg, 4.00 mmol) was added at RT and the resulting reaction mixture was stirred 4 h at RT. After completion of the reaction (monitored by LCMS), the reaction mixture was concentrated and the obtained residue was dissolved in water (5 mL). The aqueous layer was acidified using dilute HCl (1.5 N, 10 mL) and the obtained solid was filtered, dried under vacuum to afford the desired compound (1R,2S,5S)-3-((S)-2-isobutyramido-3,3-dimethylbutanoyl)-6,6-dimethyl-3-azabicyclo[3.1.0]hexane-2-carboxylic acid as an off white solid. This compound was forwarded as such to the next step without any further purification (0.45 g, crude). LCMS (ELSD): 339.4 [MH]⁺.

### Intermediate 20

### Synthesis of (1R,2S,5S)—N-(4-(cyclopropylamino)-3-hydroxy-4-oxo-1-((S)-2-oxopyrrolidin-3-yl)butan-2-yl)-3-((S)-2-isobutyramido-3,3-dimethylbutanoyl)-6,6-dimethyl-3-azabicyclo[3.1.0]hexane-2-carboxamide

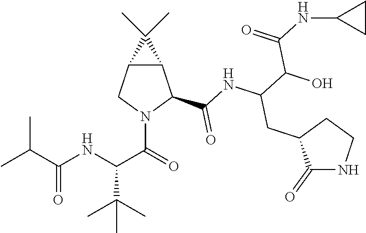 [SMILES: CC(C)C(=O)N[C@H](C(=O)N1C[C@H]2[C@@H]([C@H]1C(=O)NC(C[C@@H]1CCNC1=O)C(O)C(=O)NC1CC1)C2(C)C)C(C)(C)C]

To a suspension of (1R,2S,5S)-3-((S)-2-isobutyramido-3,3-dimethylbutanoyl)-6,6-dimethyl-3-azabicyclo[3.1.0]hexane-2-carboxylic acid (200 mg, 0.59 mmol) in DMF (3 mL) 0° C. 3-amino-N-cyclopropyl-2-hydroxy-4-((S)-2-oxopyrrolidin-3-yl)butanamide hydrochloride (164 mg, 0.59 mmol) and DIPEA (0.310 mL, 1.77 mmol) were added. Then HATU (337 mg, 0.88 mmol) was added at 0° C. and the resultant reaction mixture was stirred 2 h at RT. After completion of the reaction (monitored by UPLC-LCMS), the reaction mixture was diluted with water (5 mL) and the aqueous layer was extracted with EtOAc (3×10 mL). The combined organic layer was washed with brine (5 mL), dried over anhydrous Na₂SO₄, filtered and concentrated under reduced pressure. The resulting crude compound was purified by flash column chromatography using (silica gel: 100-200 mesh, eluent: 0-10% MeCOH in DCM as gradient) to afford the desired compound (1R,2S,5S)—N-(4-(cyclopropylamino)-3-hydroxy-4-oxo-1-((S)-2-oxopyrrolidin-3-yl)butan-2-yl)-3-((S)-2-isobutyramido-3,3-dimethylbutanoyl)-6,6-dimethyl-3-azabicyclo[3.1.0]hexane-2-carboxamide as a pale yellow gum. The mixture was forwarded as such to the next step without any further purification (0.250 g, mixture). LCMS (ELSD): 562.4 [MH]⁺.

### Example 5

### Synthesis of (1R,2S,5S)—N-[3-(cyclopropylamino)-2,3-dioxo-1-[[(3S)-2-oxopyrrolidin-3-yl]methyl]propyl]-3-[(2S)-3,3-dimethyl-2-(2-methylpropanoylamino)butanoyl]-6,6-dimethyl-3-azabicyclo[3.1.0]hexane-2-carboxamide

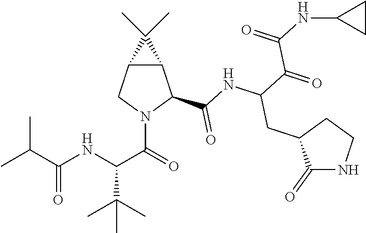 [SMILES: CC(C)C(=O)N[C@H](C(=O)N1C[C@H]2[C@@H]([C@H]1C(=O)NC(C[C@@H]1CCNC1=O)C(=O)C(=O)NC1CC1)C2(C)C)C(C)(C)C]

To a suspension of (1R,2S,5S)—N-(4-(cyclopropylamino)-3-hydroxy-4-oxo-1-((S)-2-oxopyrrolidin-3-yl)butan-2-yl)-3-((S)-2-isobutyramido-3,3-dimethylbutanoyl)-6,6-dimethyl-3-azabicyclo[3.1.0]hexane-2-carboxamide (250 mg, 0.44 mmol) in DCM (5 mL), Dess-Martin periodinane (378 mg, 0.89 mmol) was added at RT and the reaction mixture was stirred at RT for 1 h. After completion of the reaction (monitored by LCMS), the reaction mixture was quenched with saturated NaHCO₃ solution (5 mL) and the aqueous layer was extracted with DCM (3×15 mL). The organic layer was separated, washed with brine solution (10 mL), dried over anhydrous Na₂SO₄ and concentrated under reduced pressure. The compound was purified by Prep-HPLC and the fractions with desired mass peak were concentrated under reduced pressure. The obtained residue was partitioned between 10% NaHCO₃ solution (20 mL) and DCM (2×20 mL). The organic layer was dried over anhydrous Na₂SO₄, concentrated under reduced pressure and lyophilized to afford the desired compound (1R,2S,5S)—N-(4-(cyclopropylamino)-3,4-dioxo-1-((S)-2-oxopyrrolidin-3-yl)butan-2-yl)-3-((S)-2-isobutyramido-3,3-dimethylbutanoyl)-6,6-dimethyl-3-azabicyclo[3.1.0]hexane-2-carboxamide as off white solid (3.5 mg, 1.37%). LCMS (ELSD): 560.5 [MH]⁺. δ 8.76 (d, J=4.8 Hz, 1H), 8.55 (d, J=8.0 Hz, 1H), 7.79-7.73 (m, 1H), 7.61 (s, 1H), 5.18-5.03 (m, 1H), 4.39 (d, J=9.2 Hz, 1H), 4.25 (s, 1H), 4.14 (d, J=9.2 Hz, 1H), 3.83-3.80 (m, 2H), 3.52-3.51 (m, 1H), 3.20-3.17 (m, 1H), 3.10-3.00 (m, 1H), 2.75-2.70 (m, 1H), 2.64-2.54 (m, 2H), 2.25-2.13 (m, 1H), 1.90-1.80 (m, 1H), 1.70-1.60 (m, 2H), 1.50-1.45 (m, 2H), 1.25 (d, J=8.0 Hz, 4H), 1.02-0.94 (m, 13H), 0.89-0.83 (m, 2H), 0.67-0.64 (m, 2H), 0.58-0.57 (m, 2H)

Examples 3, 4, 6 and 7 were synthesised using procedures similar to those detailed above.

### Biological Data

### Construct Design of SARS-CoV-2 Mpro

The SARS CoV-2-Mpro (Main Protease/3C-like protease, UniProt ID: PODTD1) protein sequence, up to and including its autocleavage boundaries, as well as the preceding N-terminal 5 amino acid residues, including the P1 glutamine residue, were codon optimised for E. coli expression and cloned into pET26b (Merck, #US169862-3) or pGEX6P1 (Fisher Scientific, #10350355) vectors using BamHI and XhoI sites. The expression constructs thus featured a native viral N-terminal sequence, as well as a C-terminal modified 3C-protease cleavage site (LEVLFQGK), with an alternative lysine residue at the P2′ position, followed by a polyhistidine (His-8) tag.

### Protein Expression and Protein Purification

Chemically competent BL21(DE3)-RIL E. coli (Agilent, #230240) cells were transformed with the relevant coronavirus Mpro construct and grown overnight at 37° C. on LB agar plates supplemented with the appropriate antibiotics. All culture steps were performed at 37° C. unless otherwise stated. A scraping of colonies was grown in 15 mL of antibiotic supplemented LB media, for a period of approximately 2 hours, taking care not to exceed an optical density (OD) density of 2.0 as measured in a spectrophotometer at 600 nm. This preculture was used to inoculate a 500 mL expression culture: either LB media for IPTG induced expression or autoinduction superbroth media (Formedium, #AIMSB0210). In LB media, expression was induced at an OD of 0.7-1.0 by the addition of IPTG to a final concentration of 0.5 mM. The culture was then grown at 18° C. overnight. In autoinduction expression, the temperature was dropped to 18° C. once an OD of 0.7-1.0 was observed then grown overnight. The cells were harvested by centrifugation and frozen until use.

Thawed cells were resuspended into resuspension buffer: 20 mM Tris-HCl pH 8.0, 150 mM NaCl, and DNase I (Merck #4716728001) and lysed by sonication. The lysate was clarified by centrifugation at 23,000 rcf for 15 mins at 4° C. The supernatant was loaded onto 5 mL of NiNTA resin (Cytiva, #17-5248-02) at a flow-rate of 0.5 mL/min. The resin was washed with the same buffer as above containing 20 mM imidazole. Mpro protein was eluted using the same buffer containing 250 mM imidazole. The target protein was further purified using a Superdex S75 16/60 pg (GE, #GE28-9893-33) column in resuspension buffer. Protein purity was assessed by SDS-PAGE and identity confirmed by mass spectrometry. Purified protein was concentrated and frozen until later use.

### SARS-CoV-2 Mpro Enzyme Assay

The activity of SARS-Cov-2 Mpro was determined in a Fluorescence Resonance Energy Transfer (FRET)-based enzymatic assay using FRET Substrate Dabcyl-KTSAVLQSGFRKM-E (Edans)-Amide. In brief, 5 μL of test compounds (concentrations ranging from 100 μM to 0.0017 μM) was preincubated with 5 μL of 20 nM (final concentration) Mpro enzyme for 30 min at 30° C. in an assay buffer containing 20 mM HEPES, 120 mM NaCl, 0.4 mM EDTA, and 4 mM DTT and 20% glycerol. Reaction was initiated by addition of 10 μL of 20 μM (final concentration) of FRET substrate (Dabcyl-KTSAVLQSGFRKM-E (Edans)-Amide). The reaction was incubated for 1 h and the resulting fluorescent intensity was measured at Ex=360 nm/Em=490 nm at 30° C. using a SPARK 20M plate reader (Tecan). Boceprevir was used a reference standard compound. pIC₅₀ and pKi were determined using 4PL GraphPad Prism and data were represented as a mean n=2±SD.

| | SARS-CoV-2 Mpro enzyme pKi |
|---|---|
| Example | assay pKi |
| 1 | 8.4 |
| 2 | 7.7 |
| 3 | 7.2 |
| 4 | 7.1 |
| 5 | 7.8 |
| 6 | 8.0 |
| 7 | 7.2 |

### MDCK-MDR1 Permeability and P-Gp Substrate Assay

Permeability and drug efflux of certain example compounds was investigated using the following assay.

Test compound was added to either the apical side of a confluent monolayer of MDCK-MDR1 (Madin-Darby canine kidney cells stably transfected to express active P-glycoprotein) and permeability was measured by monitoring the appearance of the test compound on the basolateral side of the monolayer using LC-MS/MS. Experiments were performed in the absence and presence of a P-glycoprotein (P-gp) inhibitor elacridar (2 μM) to determine whether the compound was subject to P-gp mediated efflux.

### Procedure

MDCK-MDR1 cells obtained from the NIH (Rockville, MD, USA) were used between passage numbers 6-30. Cells were seeded onto Transwell plates at 3.4×10⁵ cells/cm². The cells were cultured in DMEM and media was changed on day 3. On day 4 or 5 the permeability study was performed. Cell culture and assay incubations were carried out at 37° C. in an atmosphere of 5% CO₂ with a relative humidity of 95%. On the day of the assay, the monolayers were prepared by rinsing both apical and basolateral surfaces twice with Hanks Balanced Salt Solution (HBSS) at the desired pH warmed to 37° C. Cells were then incubated with HBSS at the desired pH in both apical and basolateral compartments for 40 min to stabilise physiological parameters.

Test compound was diluted with assay buffer to give the final test compound concentration (typically 1 μM). Final DMSO concentration of 1% (v/v). The fluorescent integrity marker lucifer yellow was also included in the dosing solution. Analytical standards were prepared from test compound DMSO dilutions and transferred to buffer, maintaining a ≤1% (v/v) DMSO concentration. Typical assay buffer was composed of supplemented HBSS pH 7.4.

For assessment of A-B permeability, HBSS was removed from the apical compartment and replaced with test compound dosing solution or test compound plus P-gp inhibitor dosing solutions. The apical compartment insert was then placed into a companion plate containing fresh buffer (containing 51% (v/v) DMSO). At 60 min the apical compartment inserts and the companion plates were separated and apical and basolateral samples diluted for analysis. Test compound permeability was assessed in duplicate. Compounds of known permeability characteristics were run as controls on each assay plate.

Test and control compounds were quantified by LC-MS/MS cassette analysis using a 7-point calibration with appropriate dilution of the samples. The starting concentration (C₀) was determined from the dosing solution and the experimental recovery calculated from C₀ and both apical and basolateral compartment concentrations.

The permeability coefficient (Pₐₚₚ) for each compound in the absence and presence of a P-gp inhibitor was calculated from the following equation:

$${P}_{a p p} = ( \frac{dQ / dt}{{C}_{0} \times A} )$$

Where dQ/dt is the rate of permeation of the drug across the cells, C₀ is the donor compartment concentration at time zero and A is the area of the cell monolayer. C₀ was obtained from analysis of the dosing solution.

| | Mean Pₐₚₚ A-B | Mean Pₐₚₚ A-B + inhibitor |
|---|---|---|
| Example | (10⁻⁶ cm/sec) | (10⁻⁶ cm/sec) |
| 1 | 0.9 | 0.7 |
| 2 | 2.2 | 15.1 |

As demonstrated by this example, compounds having cycloalkyl such as cyclopropyl at the position “R²” of the formulae described herein were shown to provide particularly good pharmacokinetic properties. In particular, such compounds were shown to have high permeability when tested both with and without a P-gp inhibitor. These compounds were also shown to have high in vivo activity when tested in mouse and hamster models. Without wishing to be bound by the theory, this permeability may result from the increased lipophilicity of such compounds.

### Reference Examples

Also disclosed herein are the following reference compounds which were synthesised in a similar process to that described above. The details of their synthesis and activity data can be found in PCT/GB2022/050836.

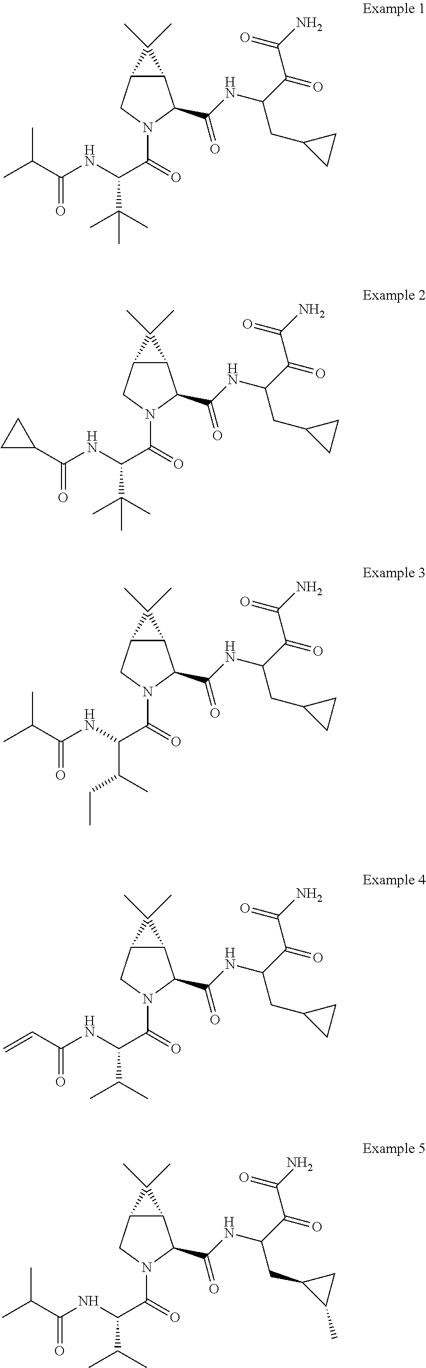 [SMILES: CC(C)C(=O)N[C@H](C(=O)N1C[C@H]2[C@@H]([C@H]1C(=O)NC(CC1CC1)C(=O)C(N)=O)C2(C)C)C(C)(C)C ; CC(C)(C)[C@H](NC(=O)C1CC1)C(=O)N1C[C@H]2[C@@H]([C@H]1C(=O)NC(CC1CC1)C(=O)C(N)=O)C2(C)C ; CC[C@@H](C)[C@H](NC(=O)C(C)C)C(=O)N1C[C@H]2[C@@H]([C@H]1C(=O)NC(CC1CC1)C(=O)C(N)=O)C2(C)C ; C=CC(=O)N[C@H](C(=O)N1C[C@H]2[C@@H]([C@H]1C(=O)NC(CC1CC1)C(=O)C(N)=O)C2(C)C)C(C)C ; CC(C)C(=O)N[C@H](C(=O)N1C[C@H]2[C@@H]([C@H]1C(=O)NC(C[C@H]1C[C@@H]1C)C(=O)C(N)=O)C2(C)C)C(C)C]

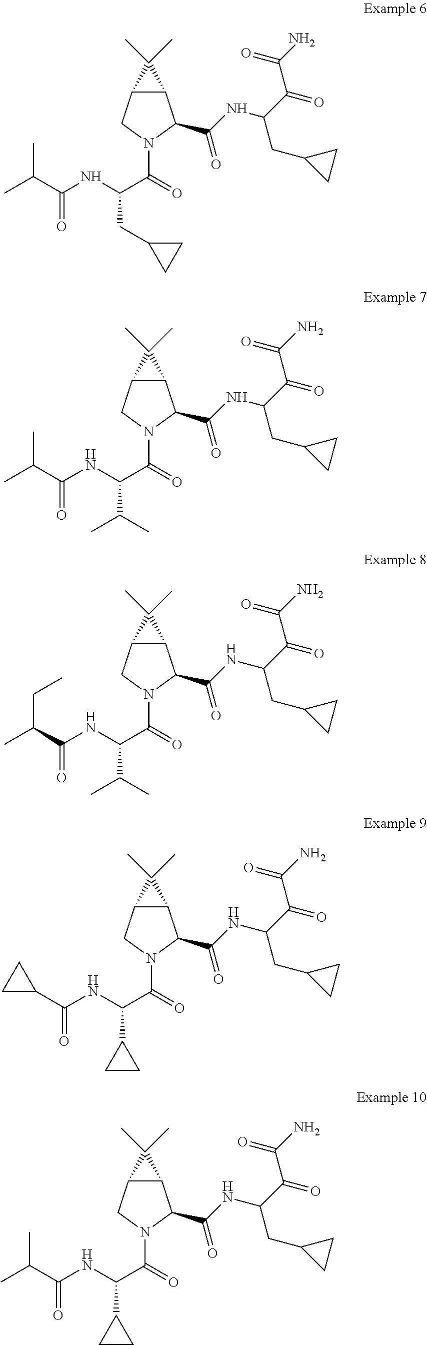 [SMILES: CC(C)C(=O)N[C@@H](CC1CC1)C(=O)N1C[C@H]2[C@@H]([C@H]1C(=O)NC(CC1CC1)C(=O)C(N)=O)C2(C)C ; CC(C)C(=O)N[C@H](C(=O)N1C[C@H]2[C@@H]([C@H]1C(=O)NC(CC1CC1)C(=O)C(N)=O)C2(C)C)C(C)C ; CC[C@H](C)C(=O)N[C@H](C(=O)N1C[C@H]2[C@@H]([C@H]1C(=O)NC(CC1CC1)C(=O)C(N)=O)C2(C)C)C(C)C ; CC1(C)[C@@H]2[C@@H](C(=O)NC(CC3CC3)C(=O)C(N)=O)N(C(=O)[C@@H](NC(=O)C3CC3)C3CC3)C[C@@H]21 ; CC(C)C(=O)N[C@H](C(=O)N1C[C@H]2[C@@H]([C@H]1C(=O)NC(CC1CC1)C(=O)C(N)=O)C2(C)C)C1CC1]

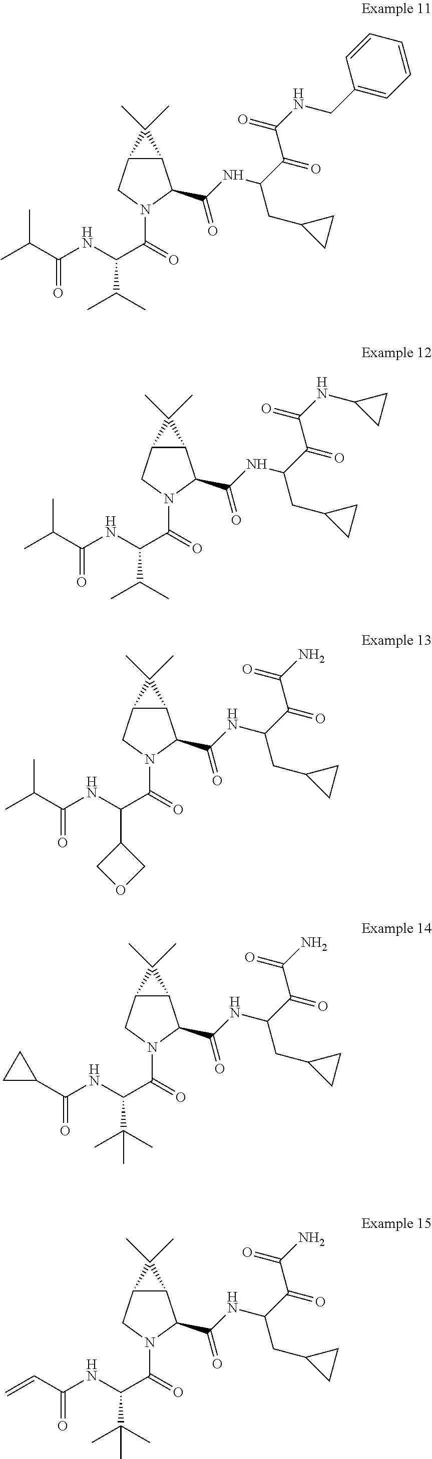 [SMILES: CC(C)C(=O)N[C@H](C(=O)N1C[C@H]2[C@@H]([C@H]1C(=O)NC(CC1CC1)C(=O)C(=O)NCc1ccccc1)C2(C)C)C(C)C ; CC(C)C(=O)N[C@H](C(=O)N1C[C@H]2[C@@H]([C@H]1C(=O)NC(CC1CC1)C(=O)C(=O)NC1CC1)C2(C)C)C(C)C ; CC(C)C(=O)NC(C(=O)N1C[C@H]2[C@@H]([C@H]1C(=O)NC(CC1CC1)C(=O)C(N)=O)C2(C)C)C1COC1 ; CC(C)(C)[C@H](NC(=O)C1CC1)C(=O)N1C[C@H]2[C@@H]([C@H]1C(=O)NC(CC1CC1)C(=O)C(N)=O)C2(C)C ; C=CC(=O)N[C@H](C(=O)N1C[C@H]2[C@@H]([C@H]1C(=O)NC(CC1CC1)C(=O)C(N)=O)C2(C)C)C(C)(C)C]

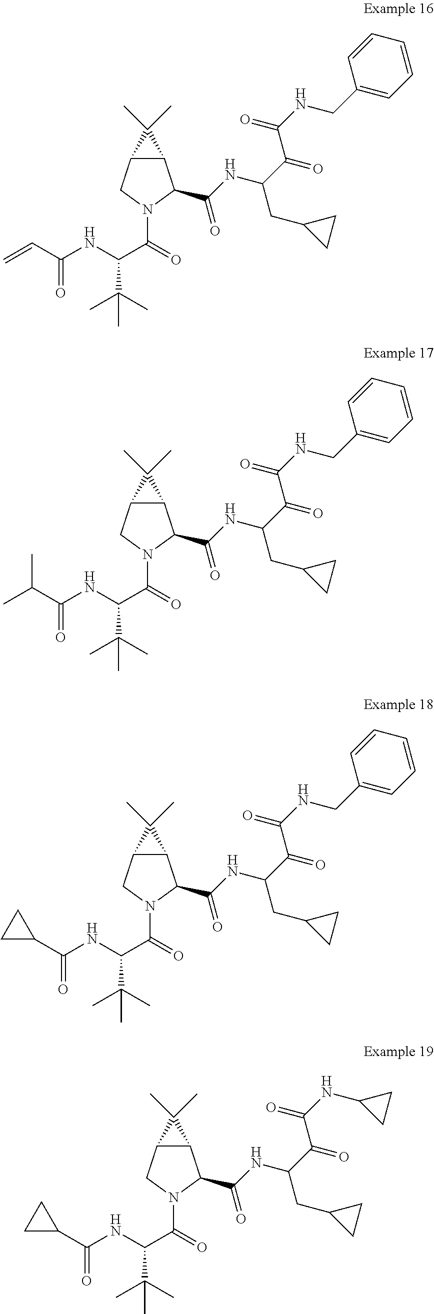 [SMILES: C=CC(=O)N[C@H](C(=O)N1C[C@H]2[C@@H]([C@H]1C(=O)NC(CC1CC1)C(=O)C(=O)NCc1ccccc1)C2(C)C)C(C)(C)C ; CC(C)C(=O)N[C@H](C(=O)N1C[C@H]2[C@@H]([C@H]1C(=O)NC(CC1CC1)C(=O)C(=O)NCc1ccccc1)C2(C)C)C(C)(C)C ; CC(C)(C)[C@H](NC(=O)C1CC1)C(=O)N1C[C@H]2[C@@H]([C@H]1C(=O)NC(CC1CC1)C(=O)C(=O)NCc1ccccc1)C2(C)C ; CC(C)(C)[C@H](NC(=O)C1CC1)C(=O)N1C[C@H]2[C@@H]([C@H]1C(=O)NC(CC1CC1)C(=O)C(=O)NC1CC1)C2(C)C]

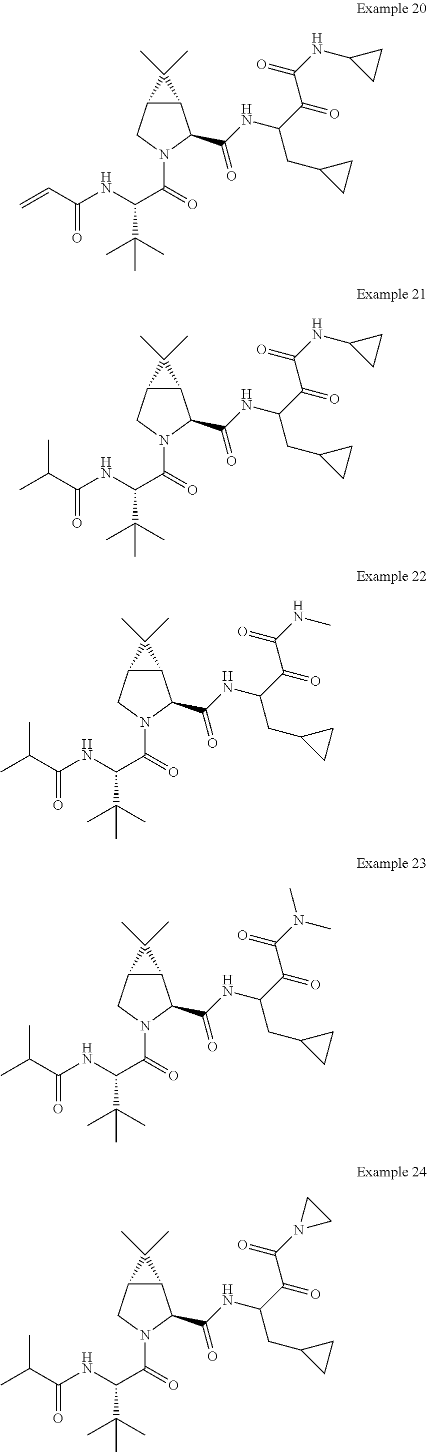 [SMILES: C=CC(=O)N[C@H](C(=O)N1C[C@H]2[C@@H]([C@H]1C(=O)NC(CC1CC1)C(=O)C(=O)NC1CC1)C2(C)C)C(C)(C)C ; CC(C)C(=O)N[C@H](C(=O)N1C[C@H]2[C@@H]([C@H]1C(=O)NC(CC1CC1)C(=O)C(=O)NC1CC1)C2(C)C)C(C)(C)C ; CNC(=O)C(=O)C(CC1CC1)NC(=O)[C@@H]1[C@@H]2[C@H](CN1C(=O)[C@@H](NC(=O)C(C)C)C(C)(C)C)C2(C)C ; CC(C)C(=O)N[C@H](C(=O)N1C[C@H]2[C@@H]([C@H]1C(=O)NC(CC1CC1)C(=O)C(=O)N(C)C)C2(C)C)C(C)(C)C ; CC(C)C(=O)N[C@H](C(=O)N1C[C@H]2[C@@H]([C@H]1C(=O)NC(CC1CC1)C(=O)C(=O)N1CC1)C2(C)C)C(C)(C)C]

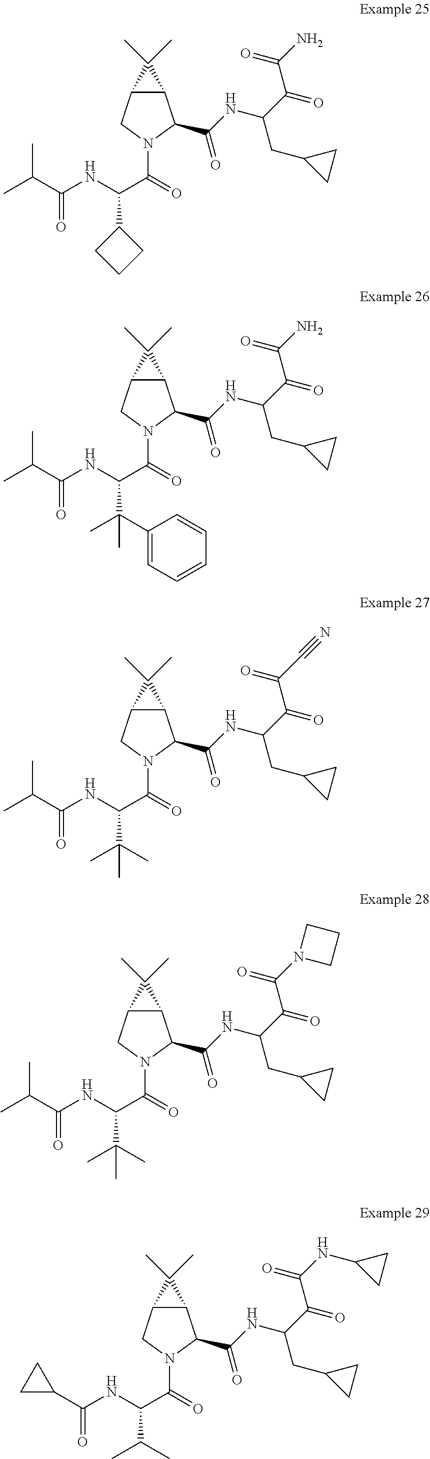 [SMILES: CC(C)C(=O)N[C@H](C(=O)N1C[C@H]2[C@@H]([C@H]1C(=O)NC(CC1CC1)C(=O)C(N)=O)C2(C)C)C1CCC1 ; CC(C)C(=O)N[C@H](C(=O)N1C[C@H]2[C@@H]([C@H]1C(=O)NC(CC1CC1)C(=O)C(N)=O)C2(C)C)C(C)(C)c1ccccc1 ; CC(C)C(=O)N[C@H](C(=O)N1C[C@H]2[C@@H]([C@H]1C(=O)NC(CC1CC1)C(=O)C(=O)C#N)C2(C)C)C(C)(C)C ; CC(C)C(=O)N[C@H](C(=O)N1C[C@H]2[C@@H]([C@H]1C(=O)NC(CC1CC1)C(=O)C(=O)N1CCC1)C2(C)C)C(C)(C)C ; CC(C)[C@H](NC(=O)C1CC1)C(=O)N1C[C@H]2[C@@H]([C@H]1C(=O)NC(CC1CC1)C(=O)C(=O)NC1CC1)C2(C)C]

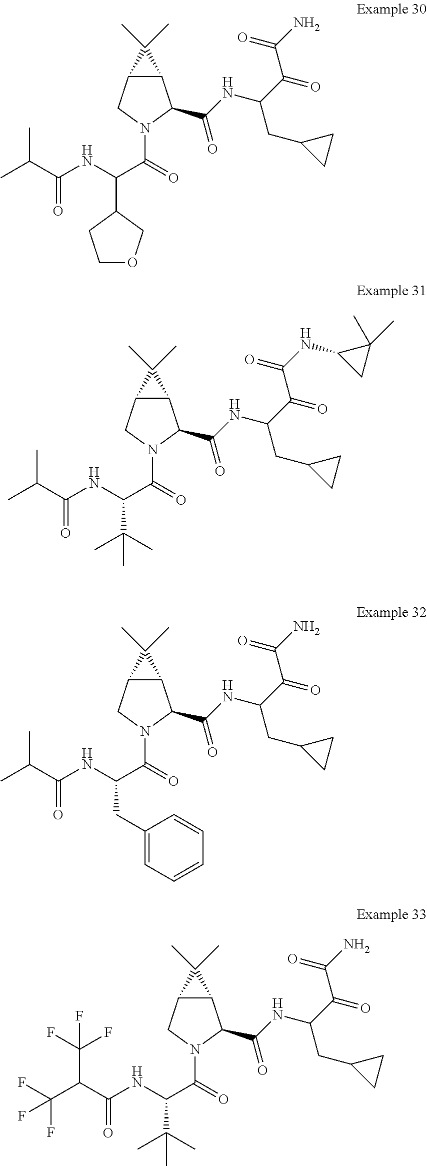

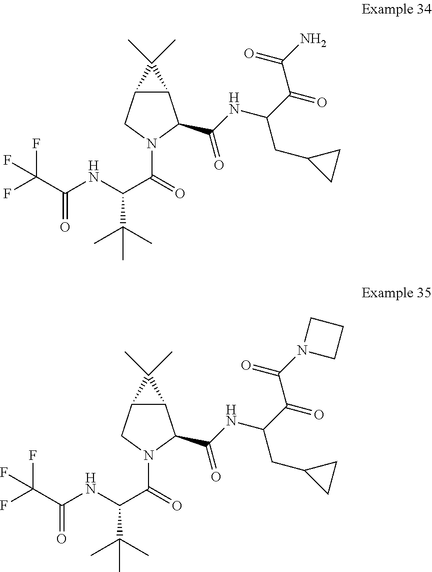 [SMILES: CC(C)(C)[C@H](NC(=O)C(F)(F)F)C(=O)N1C[C@H]2[C@@H]([C@H]1C(=O)NC(CC1CC1)C(=O)C(N)=O)C2(C)C ; CC(C)(C)[C@H](NC(=O)C(F)(F)F)C(=O)N1C[C@H]2[C@@H]([C@H]1C(=O)NC(CC1CC1)C(=O)C(=O)N1CCC1)C2(C)C]

The pKi data for these reference examples in the table below were obtained via a modified SARS-CoV-2 Mpro enzyme assay (as disclosed below), and which is set out in PCT/GB2022/050836.

### SARS-CoV-2 Mpro Enzyme Assay

The activity of SARS-Cov-2 Mpro was determined in a Fluorescence Resonance Energy Transfer (FRET)-based enzymatic assay using FRET Substrate Dabcyl-KTSAVLQSGFRKM-E (Edans)-Amide. In brief, 100 nL of test compounds (concentrations ranging from 10 μM to 0.00051 μM) was preincubated with 5 μL of 5 nM (final concentration) Mpro enzyme for 20 min at room temperature in an assay buffer containing 20 mM Tris (pH 7.5), 100 mM NaCl and 1 mM EDTA. Reaction was initiated by addition of 5 μL of 25 μM (final concentration) of FRET substrate (Dabcyl-KTSAVLQSGFRKM-E (Edans)-Amide). The resulting fluorescent intensity at Ex=360 nm/Em=490 nm was measured every 90 s over the course of 60 min at room temperature using a PHERAstar plate reader (BMG Labtech). Using MARS software (BMG Labtech), the linear portion of the reaction was selected and the rate in RFU per minute calculated. Boceprevir was used a reference standard compound. pIC₅₀ and pKi were determined using 4PL GraphPad Prism and data were represented as a mean n=2±SD. pKi values of compounds of the invention are shown in the table below.

| | SARS-CoV-2 Mpro enzyme |
|---|---|
| Ref Example No. | pKi assay pKi |
| 1 | 7.8 |
| 2 | 7.3 |
| 3 | 7.1 |
| 4 | 7.3 |
| 5 | 6.8 |
| 6 | 6.7 |
| 7 | 6.7 |
| 8 | 6.5 |
| 9 | 6.5 |
| 10 | 6.5 |
| 11 | 7.2 |
| 12 | 6.5 |
| 13 | 6.5 |
| 14 | 7.8 |
| 15 | 8.4 |
| 16 | 7.3 |
| 17 | 6.9 |
| 18 | 7.3 |
| 19 | 7.3 |
| 20 | 7.2 |
| 21 | 6.8 |
| 22 | 7.8 |
| 23 | <5.3 |
| 24 | Not tested |
| 25 | 6.6 |
| 26 | 7.4 |
| 27 | <5.3 |
| 28 | 8.2 |
| 29 | 6.6 |
| 30 | 6.8 |
| 31 | 7.0 |
| 32 | 7.2 |
| 33 | 6.8 |
| 34 | 8.4 |
| 35 | 7.4 |

These data demonstrate the efficacy of compounds having related structures to those described herein.

### Numbered Embodiments

1. A compound of Formula (I)

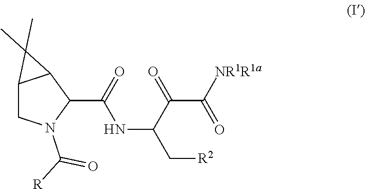

or a salt, solvate, hydrate, N-oxide or prodrug thereof, wherein:
   R¹ and R^{1a} are independently H; a C₁₋₆ saturated hydrocarbon group optionally containing a cycloalkyl group and optionally substituted with 1 to 6 halo; or a benzyl group optionally substituted with 1 to 6 halo; or R¹ and R^{1a} are linked, together with the nitrogen to which they are attached, to form a 3 to 6 membered saturated ring optionally containing an additional heteroatom and optionally substituted with 1 to 6 halo;
   R² is selected from a C₃₋₆ cycloalkyl, C₁₋₄ alkyl and saturated 3- to 6-membered heterocyclic ring, each of which are optionally substituted with one or more oxo, hydroxy and halo groups; or R² is —(CH₂)ₚCONHR⁶, or R² is —(CH₂)ₚCO₂R⁶;
   R⁶ is selected from H, C₁₋₃ alkyl, and C₁₋₃ haloalkyl;
   R represents

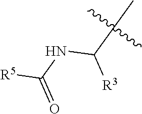

R³ is selected from
(i) a saturated group containing 1 to 6 carbon atoms and optionally containing a cycloalkyl group; or
(ii) a saturated ring containing an oxygen or nitrogen heteroatom, said saturated group or ring being optionally substituted with one or more substituents chosen from fluorine, hydroxy or methoxy; or
R³ is —CH₂aryl; —(CH₃)aryl; or —C(CH₃)₂aryl; and
R⁵ is a C₁₋₈ hydrocarbon group, optionally containing one or more rings or a double bond and which is optionally substituted with one or more groups selected from halo; cyano; hydroxy; methoxy; amino; and a cycloalkyl, heterocycloalkyl, aryl and heteroaryl; or
R represents -L-A
   wherein L is —CR⁷═CR⁸—, —CHR⁹—CHR¹⁰— or —O—CHR¹¹—; R⁷ to R¹¹ are independently H, —(CH₂)ₘCO₂R¹² or C₁₋₃ alkyl optionally substituted with 1 to 6 fluorine atoms; or R⁹ and R¹⁰ are joined to form a cyclopropyl;
   A is phenyl or heteroaryl, optionally substituted with one or more groups selected from halo, —CN, —CO₂R¹³, —OR¹³, —SO₂R¹³, —SONHR¹³, —OSO₂R¹³, —PO(R¹³)₂, —SF₅, C₁₋₃ alkyl and C₁₋₃ haloalkyl;
   R¹² and R¹³ are independently selected from H, C₁₋₃ alkyl, and C₁₋₃ haloalkyl; and
   m is independently 0 to 3;
and wherein when R² is a C₃₋₅ saturated hydrocarbon group containing a cycloalkyl group which is unsubstituted or substituted with one or more substituents chosen from fluorine or hydroxyl, then
   R represents

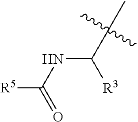

in which R⁵ is methyl, optionally substituted with one or more substituents chosen from halo; cyano; hydroxy; methoxy; amino; with R³ as defined herein; or
R represents -L-A as defined herein; and
wherein the compound of Formula (I) is not
(1R,2S,5S)—N-(4-Amino-1-cyclopropyl-3,4-dioxobutan-2-yl)-3-((S)-3,3-dimethyl-2-(2,2,2-trifluoroacetamido)butanoyl)-6,6-dimethyl-3-azabicyclo[3.1.0]hexane-2-carboxamide or a salt thereof; or
(1R,2S,5S)—N-(4-(Azetidin-1-yl)-1-cyclopropyl-3,4-dioxobutan-2-yl)-3-((S)-3,3-dimethyl-2-(2,2,2-trifluoroacetamido)butanoyl)-6,6-dimethyl-3-azabicyclo[3.1.0]hexane-2-carboxamide or a salt thereof.
   2. The compound according to embodiment 1, wherein the compound is of Formula (IA)

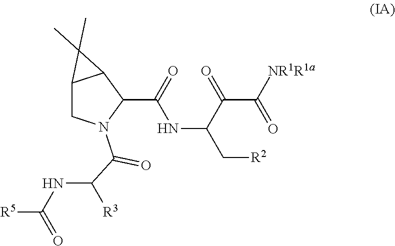

or a salt, solvate, hydrate, N-oxide or prodrug thereof, wherein the substituents are as defined in claim 1.
3. The compound according to embodiment 2 or 2, wherein R³ is selected from the group consisting of

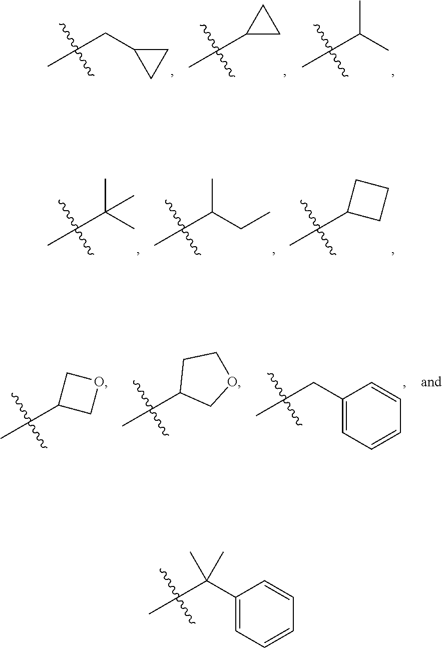

each of which are optionally substituted with one or more halo, preferably F.
4. The compound according to any preceding embodiment, wherein R³ is iso-propyl or tert-butyl.
5. The compound according to any preceding embodiment, wherein R⁵ is a saturated or unsaturated C₁₋₅ hydrocarbon group, optionally substituted with one or more halo, preferably F.
6. The compound according to any preceding embodiment, wherein R⁵ is

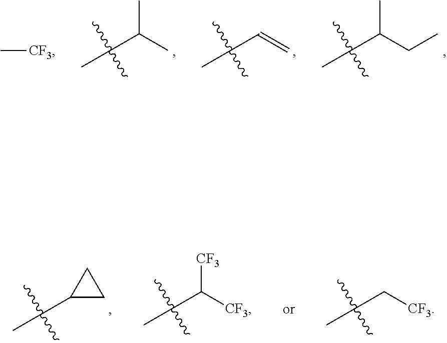

preferably —CF₃,

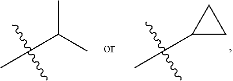

most preferably —CF₃,

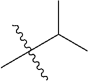 [SMILES: *C(C)C]

7. The compound according to embodiment 1, wherein the compound is of Formula (IB)

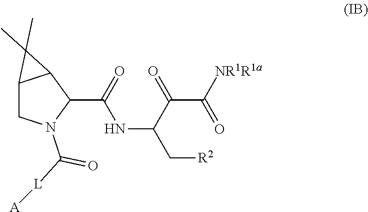

or a salt, solvate, hydrate, N-oxide or prodrug thereof, wherein the substituents are as defined in embodiment 1.
8. The compound according to any embodiment 1 or 7, wherein L is —CH═CH—,
—CH₂CH₂—, —CH₂—CH(CH₂CH₃)—, —CH₂—CH(CH₂CO₂H)—, —OCH₂—, —CH₂—CH(CH₃)—, —CH₂—CH(CF₃)—, —OCH(CH₃)—, or

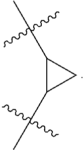 [SMILES: *C1CC1*]

9. The compound according to any of embodiments 1, 7 or 8, wherein L is —CH═CH—.
10. The compound according to any of embodiments 1, 7, 8 or 9, wherein A is phenyl or pyridinyl, each of which is optionally substituted with one or more groups selected from halo, hydroxy and C₁₋₃ alkyl.
11. The compound according to any of embodiments 1, 7, 8, 9 or 10, wherein A is selected from

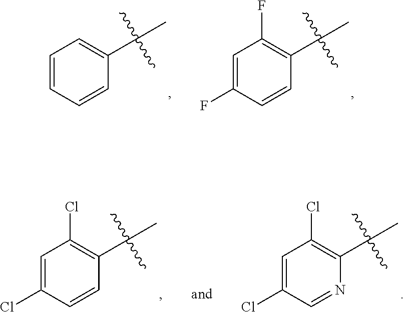 [SMILES: *c1ccccc1 ; *c1ccc(F)cc1F ; *c1ccc(Cl)cc1Cl ; *c1ncc(Cl)cc1Cl]

12. The compound according to any preceding embodiment, wherein
(i) R¹ is H or a C₁₋₆ saturated hydrocarbon group optionally containing a cycloalkyl group and optionally substituted with 1 to 6 halo, preferably F, and/or
(ii) R^{1a} is H.
preferably R¹ is H, methyl or cyclopropyl, and R^{1a} is H.
13. The compound according to any one of embodiments 1 to 11, wherein R¹ and R^{1a} are linked, together with the nitrogen to which they are attached, to form a 3 to 6 membered saturated ring optionally containing an additional heteroatom and optionally substituted with 1 to 6 halo, preferably F.
14. The compound according to any preceding embodiment, wherein R² is C₃₋₆ cycloalkyl, or a saturated 5- or 6-membered heterocyclic ring, optionally substituted with one or more oxo, hydroxy and halo.
15. The compound according to any preceding embodiment, wherein R² is selected from the group consisting of

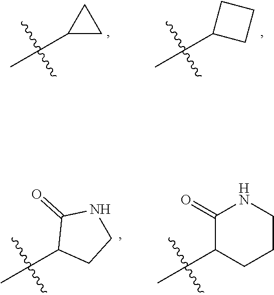

each of which is optionally substituted by one or more halo, preferably F.
16. The compound according to embodiment 1, wherein the compound is selected from the group consisting of
N-(4-(cyclopropylamino)-3,4-dioxo-1-(2-oxopyrrolidin-3-yl)butan-2-yl)-3-(3,3-dimethyl-2-(2,2,2-trifluoroacetamido)butanoyl)-6,6-dimethyl-3-azabicyclo[3.1.0]hexane-2-carboxamide;
N-[1-(cyclopropylmethyl)-3-(methylamino)-2,3-dioxo-propyl]-3-[3,3-dimethyl-2-[(2,2,2-trifluoroacetyl)amino]butanoyl]-6,6-dimethyl-3-azabicyclo[3.1.0]hexane-2-carboxamide;
N-[3-amino-1-(cyclopropylmethyl)-2,3-dioxo-propyl]-6,6-dimethyl-3-[3-methyl-2-[(2,2,2-trifluoroacetyl)amino]butanoyl]-3-azabicyclo[3.1.0]hexane-2-carboxamide;
N-[3-amino-1-(cyclobutylmethyl)-2,3-dioxo-propyl]-3-[3,3-dimethyl-2-[(2,2,2-trifluoroacetyl)amino]butanoyl]-6,6-dimethyl-3-azabicyclo[3.1.0]hexane-2-carboxamide;
N-[3-(cyclopropylamino)-2,3-dioxo-1-[[2-oxopyrrolidin-3-yl]methyl]propyl]-3-[3,3-dimethyl-2-(2-methylpropanoylamino)butanoyl]-6,6-dimethyl-3-azabicyclo[3.1.0]hexane-2-carboxamide;
N-[3-(cyclopropylamino)-2,3-dioxo-1-[[2-oxo-3-piperidyl]methyl]propyl]-3-[3,3-dimethyl-2-[(2,2,2-trifluoroacetyl)amino]butanoyl]-6,6-dimethyl-3-azabicyclo[3.1.0]hexane-2-carboxamide; and
N-(4-(cyclopropylamino)-3,4-dioxo-1-(2-oxopyrrolidin-3-yl)butan-2-yl)-3-(3-(2,4-difluorophenyl)acryloyl)-6,6-dimethyl-3-azabicyclo[3.1.0]hexane-2-carboxamide,
   or salt, solvate, hydrate, N-oxide or prodrug thereof.
   17. A pharmaceutical composition comprising a compound as defined in any preceding embodiment, and a pharmaceutically acceptable excipient.
   18. A compound as defined in any one of embodiments 1 to 16, or a pharmaceutical composition as defined in embodiment 17, for use as a medicament.
   19. A compound as defined in any one of embodiments 1 to 16, or a pharmaceutical composition as defined in embodiment 17, for use in the treatment of SARS-CoV-2 or in the treatment of disorders associated with SARS-CoV-2.
   20. Use of a compound as defined in any one of embodiments 1 to 16 in the manufacture of a medicament for the treatment of SARS-CoV-2 or a disorder associated with SARS-CoV-2.
   21. A method of treating a disease or disorder susceptible to SARS-CoV-2 Mpro inhibition in a subject in need thereof, said method comprising administering to said subject a pharmaceutically effective amount of a compound as defined in any one of embodiments 1 to 16.
   22. A method of treating SARS-CoV-2, or a disorder associated with SARS-CoV-2, in a subject in need thereof, said method comprising administering to said subject a pharmaceutically effective amount of the compound as defined in any one of embodiments 1 to 16.

1. A compound of Formula (I′) 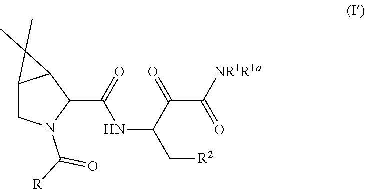
or a salt, solvate, hydrate, N-oxide or prodrug thereof, wherein:
R¹ and R^{1a} are independently H; a C₁₋₆ saturated hydrocarbon group optionally containing a cycloalkyl group and optionally substituted with 1 to 6 halo; or a benzyl group optionally substituted with 1 to 6 halo;
or R¹ and R^{1a} are linked, together with the nitrogen to which they are attached, to form a 3 to 6 membered saturated ring optionally containing an additional heteroatom and optionally substituted with 1 to 6 halo;
R² is C₃₋₆ cycloalkyl which is optionally substituted with one or more oxo, hydroxy and halo groups;
R represents 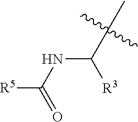
R³ is selected from
(i) a saturated group containing 1 to 6 carbon atoms and optionally containing a cycloalkyl group; or
(ii) a saturated ring containing an oxygen or nitrogen heteroatom, said saturated group or ring being optionally substituted with one or more substituents chosen from fluorine, hydroxy or methoxy; or
R³ is —CH₂aryl; —CH(CH₃)aryl; or —C(CH₃)₂aryl; and
R⁵ is a C₁₋₈ hydrocarbon group, optionally containing one or more rings or a double bond and which is optionally substituted with one or more groups selected from halo; cyano; hydroxy; methoxy; amino; and a cycloalkyl, heterocycloalkyl, aryl and heteroaryl; or
R represents -L-A
wherein L is —CR⁷═CR⁸—, —CHR⁹—CHR¹⁰— or —O—CHR¹¹—;
R⁷ to R¹¹ are independently H, —(CH₂)ₘCO₂R¹² or C₁₋₃ alkyl optionally substituted with 1 to 6 fluorine atoms; or
R⁹ and R¹⁰ are joined to form a cyclopropyl;
A is phenyl or heteroaryl, optionally substituted with one or more groups selected from halo, —CN, —CO₂R¹³,
—OR¹³, —SO₂R¹³, —SONHR¹³, —OSO₂R¹³, —PO(R¹³)₂, —SF₅, C₁₋₃ alkyl and C₁₋₃ haloalkyl;
R¹² and R¹³ are independently selected from H, C₁₋₃ alkyl, and C₁₋₃ haloalkyl; and
m is independently 0 to 3;
and wherein when R² is a C₃₋₅ saturated hydrocarbon group containing a cycloalkyl group which is unsubstituted or substituted with one or more substituents chosen from fluorine or hydroxyl, then
R represents 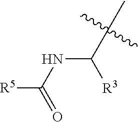
in which R⁵ is methyl, optionally substituted with one or more substituents chosen from halo; cyano; hydroxy; methoxy; and amino; with R³ as defined herein; or
R represents -L-A as defined herein; and
wherein the compound of Formula (I′) is not
(1R,2S,5S)—N-(4-Amino-1-cyclopropyl-3,4-dioxobutan-2-yl)-3-((S)-3,3-dimethyl-2-(2,2,2-trifluoroacetamido)butanoyl)-6,6-dimethyl-3-azabicyclo[3.1.0]hexane-2-carboxamide or a salt thereof; or
(1R,2S,5S)—N-(4-(Azetidin-1-yl)-1-cyclopropyl-3,4-dioxobutan-2-yl)-3-((S)-3,3-dimethyl-2-(2,2,2-trifluoroacetamido)butanoyl)-6,6-dimethyl-3-azabicyclo[3.1.0]hexane-2-carboxamide or a salt thereof.

2. The compound as claimed in claim 1, wherein the compound is of Formula (IA) 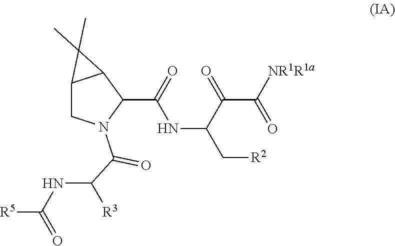
or a salt, solvate, hydrate, N-oxide or prodrug thereof, wherein the substituents are as defined in claim 1.

3. The compound as claimed in claim 1, wherein R³ is selected from the group consisting of: 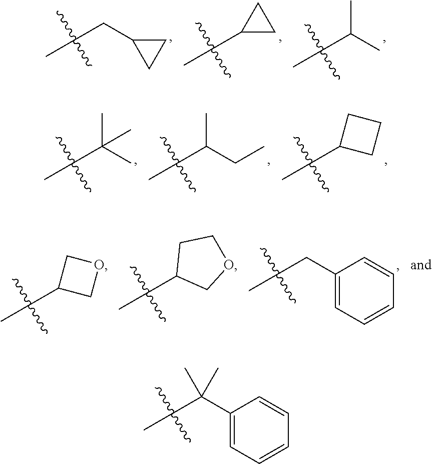
each of which are optionally substituted with one or more halo.

4. The compound as claimed in claim 1, wherein R³ is iso-propyl or tert-butyl.

5. The compound as claimed in claim 1, wherein R⁵ is a saturated or unsaturated C₁₋₅ hydrocarbon group, optionally substituted with one or more halo.

6. The compound as claimed in claim 1, wherein R⁵ is
—CF₃, 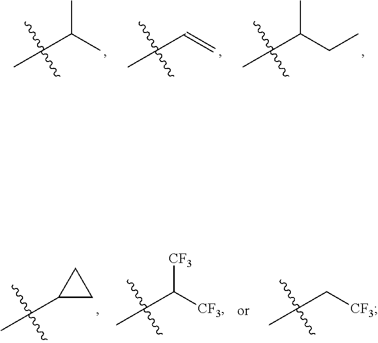
or
R⁵ is —CF₃, 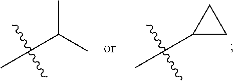
or
R⁵ is —CF₃, or

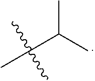 [SMILES: *C(C)C]

7. The compound as claimed in claim 1, wherein the compound is of Formula (IB) 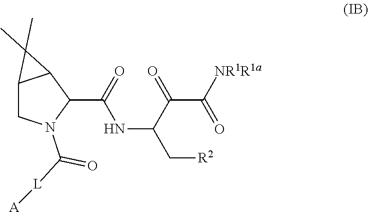
or a salt, solvate, hydrate, N-oxide or prodrug thereof, wherein the substituents are as defined in claim 1.

8. The compound as claimed in claim 1, wherein L is —CH═CH—, —CH₂CH₂—, —CH₂—CH(CH₂CH₃)—, —CH₂—CH(CH₂CO₂H)—, —OCH₂—, —CH₂—CH(CH₃)—, —CH₂—CH(CF₃)—, —OCH(CH₃)—, or

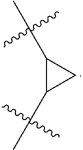 [SMILES: *C1CC1*]

9. The compound as claimed in claim 1, wherein L is —CH═CH—.

10. The compound as claimed in claim 1, wherein A is phenyl or pyridinyl, each of which is optionally substituted with one or more groups selected from halo, hydroxy, and C₁₋₃ alkyl.

11. The compound as claimed in claim 1, wherein A is selected from

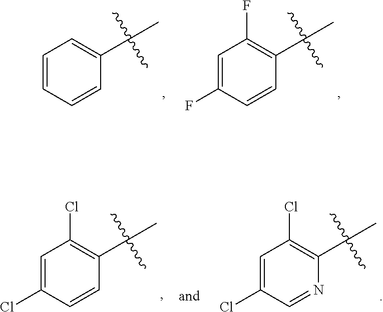 [SMILES: *c1ccccc1 ; *c1ccc(F)cc1F ; *c1ccc(Cl)cc1Cl ; *c1ncc(Cl)cc1Cl]

12. The compound as claimed in claim 1, wherein at least one of:
(i) R¹ is H or a C₁₋₆ saturated hydrocarbon group optionally containing a cycloalkyl group and optionally substituted with 1 to 6 halo;
(ii) R¹ is H, methyl, or cyclopropyl; and
(iii) R^{1a} is H.

13. The compound as claimed in claim 1, wherein R¹ and R^{1a} are linked, together with the nitrogen to which they are attached, to form a 3 to 6 membered saturated ring optionally containing an additional heteroatom and optionally substituted with 1 to 6 halo.

14. The compound as claimed in claim 1, wherein R² is selected from the group consisting of: 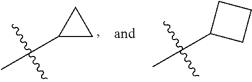
each of which is optionally substituted by one or more halo.

15. A compound selected from the group consisting of:
N-[1-(cyclopropylmethyl)-3-(methylamino)-2,3-dioxo-propyl]-3-[3,3-dimethyl-2-[(2,2,2-trifluoroacetyl)amino]butanoyl]-6,6-dimethyl-3-azabicyclo[3.1.0]hexane-2-carboxamide;
N-[3-amino-1-(cyclopropylmethyl)-2,3-dioxo-propyl]-6,6-dimethyl-3-[3-methyl-2-[(2,2,2-trifluoroacetyl)amino]butanoyl]-3-azabicyclo[3.1.0]hexane-2-carboxamide;
N-[3-amino-1-(cyclobutylmethyl)-2,3-dioxo-propyl]-3-[3,3-dimethyl-2-[(2,2,2-trifluoroacetyl)amino]butanoyl]-6,6-dimethyl-3-azabicyclo[3.1.0]hexane-2-carboxamide;
N-[3-(cyclopropylamino)-2,3-dioxo-1-[[2-oxopyrrolidin-3-yl]methyl]propyl]-3-[3,3-dimethyl-2-(2-methylpropanoylamino)butanoyl]-6,6-dimethyl-3-azabicyclo[3.1.0]hexane-2-carboxamide;
N-[3-(cyclopropylamino)-2,3-dioxo-1-[[2-oxo-3-piperidyl]methyl]propyl]-3-[3,3-dimethyl-2-[(2,2,2-trifluoroacetyl)amino]butanoyl]-6,6-dimethyl-3-azabicyclo[3.1.0]hexane-2-carboxamide; and
N-(4-(cyclopropylamino)-3,4-dioxo-1-(2-oxopyrrolidin-3-yl)butan-2-yl)-3-(3-(2,4-difluorophenyl)acryloyl)-6,6-dimethyl-3-azabicyclo[3.1.0]hexane-2-carboxamide,
or salt, solvate, hydrate, N-oxide or prodrug thereof.

16. A pharmaceutical composition comprising a compound as defined in claim 1, and a pharmaceutically acceptable excipient.

17. The pharmaceutical composition according to claim 16, further comprising one or more drugs which block cypP450 mediated metabolism.

18. The pharmaceutical composition of claim 17, wherein the one or more drugs which block CYP450 mediated metabolism are selected from ritonavir, lopinavir, or a combination thereof.

19. (canceled)

20. (canceled)

21. A method of treating a disease or disorder susceptible to SARS-CoV-2 Mpro inhibition in a subject in need thereof, said method comprising administering to said subject a pharmaceutically effective amount of a compound as defined in claim 1.

22. A method of treating SARS-CoV-2, or a disorder associated with SARS-CoV-2, in a subject in need thereof, said method comprising administering to said subject a pharmaceutically effective amount of the compound as defined in claim 1.

23. The method according to claim 21 wherein the compound is administered in combination with one or more drugs which block cypP450 mediated metabolism.

24. The method of claim 23, wherein the one or more drugs which block CYP450 mediated metabolism are selected from ritonavir, lopinavir, or a combination thereof
